(19) United States
(12) Patent Application Publication
(10) Pub. No.: US 2026/0347792 A1
(43) Pub. Date: Nov. 5, 2026

(54) Advanced Alveolar Epithelial Organoids

(71) Applicant: Children's Hospital Medical Center, Cincinnati, OH (US)

(72) Inventors: William ZACHARIAS, Cincinnati, OH (US); Andrea TOTH, Cincinnati, OH (US)

(73) Assignee: Children's Hospital Medical Center, Cincinnati, OH (US)

(21) Appl. No.: 19/105,887

(22) PCT Filed: Aug. 28, 2023
(86) PCT No.: PCT/US2023/031312

§ 371 Date: Feb. 24, 2025

Related U.S. Application Data
(60) Provisional application No. 63/402,056, filed on Aug. 29, 2022.

(51) Int. Cl. C12N 5/071

(52) U.S. Cl. CPC C12N 5/0688; C12N 2500/24; C12N 2500/38; C12N 2500/84; C12N 2501/11; C12N 2501/33; C12N 2501/727; C12N 2502/1323; C12N 2503/04

(57) ABSTRACT

Disclosed are alveolar organoids which recapitulate important aspects of in vivo lung epithelial regeneration, providing a tractable model to dissect regenerative processes. Clonal expansion of single AEPs generated complex alveolar organoids with extensive internal organization. Whole mount immunohistochemistry of these organoids showed properly patterned, polarized, and functional AT1 and AT2 cells surrounding numerous alveolar-like cavities with minimal structural contribution from mesenchymal cells, implying extensive cell autonomous regenerative function encoded in adult AEPs.

### CROSS-REFERENCE TO RELATED APPLICATIONS

This application is a National Stage Application from PCT/US2023/031312, filed Aug. 28, 2023, which claims priority to and benefit of U.S. Provisional Patent Application No. 63/402,056, filed Aug. 29, 2022. The contents of each are incorporated in their entirety for all purposes.

### STATEMENT REGARDING FEDERALLY SPONSORED R&D

This invention was made with government support under HL140178 awarded by the National Institutes of Health. The government has certain rights in the invention.

### BACKGROUND OF THE INVENTION

The pulmonary alveolar gas exchange surface is frequently challenged by pathogens, environmental toxins, and inhaled irritants such as cigarette smoke. These challenges cause chronic, recurrent stress and/or injury to the epithelial, endothelial, and mesenchymal lineages that constitute the alveolus, and homeostatic turnover and regenerative capacity in the alveolus must be sufficient to maintain adequate oxygenation and ventilation throughout life. Therefore, extensive recent attention has focused on epithelial progenitor capacity and plasticity in repair of the alveolar epithelium—a topic whose importance has been further emphasized by the COVID-19 pandemic, which has led to significant lung injury in a large proportion of the global population in under three years. Given the dearth of therapies to promote alveolar epithelial regeneration, models to define epithelial cell relationships and identify pro-regenerative pathways are an area of high priority research interest.

Multiple lung organoid approaches have been reported in recent years, derived both from primary lung epithelium and induced-pluripotent stem cells (iPSC)-. Several themes emerged from these reports. First, the term “lung organoid” encompasses a broad, heterogenous set of cultures with different compositions and morphologies. Second, advancements in purity of epithelial components and/or removal of mesenchymal supportive cells have been reported, generally at the expense of complexity. Co-culture systems are characteristically higher in cellular heterogeneity, which is an advantage in replicating the complex cellular composition of the alveolus, but the reproducibility of these co-cultures has been challenged. Third, while iPSC-derived alveolar cells have advanced understanding of human alveolar type 2 (AT2) differentiation and biology, it is difficult to model complex adult lung epithelial phenotypes and pathologies using human iPSC cultures. Finally, lung regeneration involves complex in vivo morphogenesis occurring in tandem with cellular differentiation—a major barrier to building an “alveolus in a dish” is the lack of morphological similarity between in vitro and in vivo models. These challenges have limited progression of organoid cultures as a method for studying alveolar regeneration in vitro.

### SUMMARY OF THE INVENTION

Lung epithelial regeneration after acute injury requires coordination of extensive cellular and molecular processes controlling proliferation and differentiation of specialized alveolar cells to pattern the morphologically complex alveolar gas exchange surface. Here, we report a refined primary murine alveolar organoid assay which recapitulates important aspects of in vivo lung epithelial regeneration, providing a tractable model to dissect regenerative processes. Clonal expansion of single AEPs generated complex alveolar organoids with extensive structural maturation and organization. Whole mount immunohistochemistry of these organoids showed properly patterned, polarized, and functional AT1 and AT2 cells surrounding numerous alveolar-like cavities with minimal structural contribution from mesenchymal cells, implying extensive cell autonomous regenerative function encoded in adult AEPs. Leveraging a time series of paired scRNAseq and scATACseq analysis, we defined the AEP state at single cell resolution and use this to show that AEPs give rise to alveolar cell states reported in multiple published in vivo injury and organoid datasets. Further, we defined two distinct AEP to AT1 intermediate states: a widely reported transitional state defined by cell stress markers (Krt8+/PATS/DATP/ADI cells, also referred to as a “Krt8+ stressed transitional state”) and a second state defined by differential receptivity to cellular signaling pathways important in AT1 cell differentiation. Transcriptional regulatory network (TRN) analysis demonstrates that these AT1 transition states are driven by distinct regulatory networks controlled in part by differential activity of the lung master regulatory factor Nkx2-1, which was absent in the TRN for Krt8+ cells. Genetic ablation of Nkx2-1 in AEP-derived organoids causes irreversible transition to a proliferative stressed Krt8+ state (Krt8+/PATS/DATP/ADI-like state) by disorganized, uncontrolled growth. Finally, AEP-specific deletion of Nkx2-1 in adult mice using a Tfcp211^{CreERT2} mouse line leads to rapid, irreversible loss of AEP state, clonal expansion, and disorganization of alveolar structure, (optionally with loss of proliferation). Together, these data provide new insight into lineage hierarchies in lung development and implicate dynamic epigenetic maintenance via lineage transcription factors as central to control of facultative progenitor activity in AEPs.

Embodiments of the present disclosure include the following numbered embodiments:
1. A method of making an alveolar epithelial progenitor cell (AEP)-derived organoid (AEP-O), the method comprising coculturing Wnt-responsive alveolar type 2 cells (AEP cells) and mesenchyme cells.
2. The method of embodiment 1, wherein the mesenchyme cells are fibroblast cells.
3. The method of any one of the preceding embodiments, wherein the mesenchyme cells are alveolar fibroblasts.
4. The method of any one of the preceding embodiments, wherein the mesenchyme cells are from P28 wild type C57BL/6 mice, optionally at passage 3-4.
5. The method of any one of the preceding embodiments, wherein the AEP cells are FACS sorted to select CD31⁻/CD45⁻/CD326⁺ (EpCAM⁺) cells, wherein optionally the AEP cells are TdTomato⁺.
6. The method of any one of the preceding embodiments, wherein the AEP cells are from Axin2^{CreERT2-tDT} mice.
7. The method of any one of the preceding embodiments, wherein the AEP and mesenchyme cells are cocultured in a ratio of AEP to mesenchyme that is, or is about, 2:1, 1:1, 1:2, 1:5, 1:10, 1:15, 1:20, 1:50, or a range defined by any two of the preceding values, optionally 2:1-1:50, 1:2-1:20, 1:5-1:15, or 1:10.
8. The method of any one of the preceding embodiments, wherein about 1,000, 2,000, 3,000, 4,000, 5,000, 6,000, 7,000, 8,000, 9,000, or 10,000, or a range defined by any two of the preceding values, of sorted AEPs are cocultured with about 10K, 20K, 30K, 40K, 50K, 60, 70K, 80K, 90K, or 100K, or a range defined by any two of the preceding values, mesenchymal cells.
9. The method of any one of the preceding embodiments, wherein the coculturing is in a small airway epithelial cell growth basal medium (Lonza, CC-3119) or equivalent media.
10. The method of any one of the preceding embodiments, wherein the coculturing is in a media supplemented with BPE, Insulin, Retinoic Acid, Transferrin, and hEGF.
11. The method of any one of the preceding embodiments, wherein the coculturing is in a media supplemented with heat inactivated fetal bovine serum, optionally at a final concentration of about 1-10%, 2-8%, 3-7%, or 5%.
12. The method of any one of the preceding embodiments, wherein the coculturing is in a media comprising an extracellular membrane matrix.
13. The method of any one of the preceding embodiments, wherein the coculturing is in a media comprising Matrigel.
14. The method of any one of the preceding embodiments, wherein the coculturing is in a media comprising an extracellular membrane matrix, wherein the media and the extracellular membrane matrix are combined in a ratio of 10:1, 5:1, 3:1, 2:1, 1:1, 1:2, 1:3, 1:5, or 1:10, or a range defined by any two of the preceding values.
15. The method of any one of the preceding embodiments, wherein the coculturing is in a transwell.
16. The method of any one of the preceding embodiments, wherein the coculturing is in a transwell, wherein media supplemented with a ROCK inhibitor is added beneath the transwell.
17. The method of any one of the preceding embodiments, wherein the coculturing is in a transwell, wherein media supplemented with ROCK Inhibitor Y-27632 dihydrochloride is added beneath the transwell, optionally at a final concentration of about 0.001-0.1, 0.005-0.05, or 0.01 mM.
18. The method of any one of the preceding embodiments, wherein the coculturing is in a transwell, wherein media supplemented with a ROCK inhibitor is added beneath the transwell, and wherein the coculture is incubated in the presence of the ROCK inhibitor for about 36-60, or 48 hours.
19. The method of any one of the preceding embodiments, wherein the coculturing is in a transwell, wherein media supplemented with a ROCK inhibitor is added beneath the transwell, and wherein the coculture is incubated in the presence of the ROCK inhibitor for about 36-60, or 48 hours, and wherein thereafter the media does not contain a ROCK inhibitor.
20. The method of any one of the preceding embodiments, wherein the coculturing is for a period of, or of at least, about 1, 2, 3, 4, 5, 6, 7, or 8 weeks, or a range defined by any two of the preceding values, optionally 1-8, 2-8, 3-8, 2-6, 2-5, or 3-5 weeks.
21. The method of any one of the preceding embodiments, wherein the AEP-O comprises AEP cells, AT1 cells, and AT2 cells, optionally wherein the AEP, AT1 and AT2 cells express AEP, AT1, and AT2 cell markers, respectively.
22. The method of any one of the preceding embodiments, wherein the AEP-O comprises RAGE⁺ AT1 cells.
23. The method of any one of the preceding embodiments, wherein the AEP-O comprises cavities.
24. The method of any one of the preceding embodiments, wherein the AEP-O comprises cavities forming alveolar-like structures.
25. The method of any one of the preceding embodiments, wherein the AEP-O comprises mature and/or polarized AT1 cells within the central portion of the organoid, optionally wherein AT2 cells are intermixed with the mature and/or polarized AT1 cells.
26. The method of any one of the preceding embodiments, wherein the AEP-O comprises cavities with epithelial lining that comprises AT2 cells.
27. The method of any one of the preceding embodiments, wherein the AEP-O comprises cavities with epithelial lining that comprises AT2 cells containing lamellar bodies with the apical surface directed towards the internal lumen.
28. The method of any one of the preceding embodiments, wherein the AEP-O comprises active surfactant secretion.
29. The method of any one of the preceding embodiments, wherein the AEP-O comprises mesenchymal cells, optionally fibroblasts.
30. The method of any one of the preceding embodiments, wherein the AEP-O does not comprise mesenchymal cells, optionally fibroblasts.
31. The method of any one of the preceding embodiments, wherein the AEP-O does not comprise fibillar collagen type I and/or type II.
32. The method of any one of the preceding embodiments, wherein mesenchymal cells, optionally fibroblasts, are near and/or adjacent to the AEP-O.
33. The method of any one of the preceding embodiments, wherein the AEP-O is cultured in a container comprising a monolayer of mesenchymal cells, optionally fibroblasts.
34. The method of any one of the preceding embodiments, wherein the AEP-O comprises immune cells.
35. The method of any one of the preceding embodiments, wherein the AEP-O does not comprise immune cells.
36. The method of any one of the preceding embodiments, wherein immune cells are near and/or adjacent to the AEP-O.
37. The method of any one of the preceding embodiments, wherein the AEP-O are cultured in a container comprising a monolayer of fibroblasts comprising immune cells.
38. The method of any one of the preceding embodiments, wherein the AEP-O comprises AT1 cells expressing WNT ligands and/or PDGF ligands.
39. The method of any one of the preceding embodiments, wherein the AEP-O comprises AT2 cells that are WNT-responsive.
40. The method of any one of the preceding embodiments, wherein the mesenchymal cells are WNT-responsive, PDGFRα⁺, express HGF, express non-canonical WNT, and/or express FGF ligands.
41. The method of any one of the preceding embodiments, wherein the AEP-O comprises AEP cells expressing one or more of the AEP-enriched markers Id2, Ctnnb1, Lrp5, Lrp2, Napsa, Bex2, Hdc, and Fgfr2.
42. The method of any one of the preceding embodiments, wherein the AEP-O comprises AEP cells expressing high levels of cycle genes (pAEPs).
43. The method of any one of the preceding embodiments, wherein the AEP-O comprises AT2tr cells, optionally comprising high level expression of glutathione pathway genes and a shift towards lipid metabolism.
44. The method of any one of the preceding embodiments, wherein the AEP-O comprises mature AT2 cells expressing one or more markers selected from Sftpa1, Lys2, Sftpc and Sftpb.
45. The method of any one of the preceding embodiments, wherein the AEP-O comprises Krt8⁺ transition cells (Krt8⁺) expressing one or more markers selected from Krt8, Lgals3, Tp53, Nupr1, Ddit3, and Cldn4, or optionally one or more markers selected from Krt8, Lgals3, Tp53, and Cldn4.
46. The method of any one of the preceding embodiments, wherein the AEP-O comprises AT1 transition (AT1tr) cells expressing one or more markers selected from Hes1 and Igfbp7.
47. The method of any one of the preceding embodiments, wherein the AEP-O comprises a modification reducing or eliminating expression of Nkx2-1.
48. The method of any one of the preceding embodiments, wherein the AEP-O comprises AEPs harboring a R26R-lox-stop-lox-EYFP allele.
49. The method of any one of the preceding embodiments, wherein the method comprises infecting AEPs with AAV6.2FF-Cre, optionally after FACS sorting and prior to coculturing with mesenchyme cells.
50. The method of any one of the preceding embodiments, wherein the AEPs are from Axin2^{CreERT2-Tdt}× Rosa-EYFP× Nkx2-1^{flox/flox}×animals.
51. The method of any one of the preceding embodiments, wherein the AEPs are from Tfcp211^{CreERT2}×R26R^{EYFP}×Nkx2-1^{flox/flox}×animals.
52. The method of any one of the preceding embodiments, wherein the AEPs are from Nkx2-1 knockout animals.
53. The method of any one of the preceding embodiments, wherein the AEP-O comprises at least one alveolar-like cavity filled with debris, a pseudostratrified epithelial lining, and/or a glandular-like appearance.
54. The method of any one of the preceding embodiments, wherein the AEP-O does not comprise substantial expression of one or more foregut endoderm markers selected from Sox2, Sox9, Cdx2, Gata4, and Pdx1.
55. An AEP-O made by the method of any one of the preceding embodiments.
56. An AEP-O comprising AEP cells, AT1 cells, and AT2 cells, optionally wherein the AEP, AT1 and AT2 cells express AEP, AT1, and AT2 cell markers, respectively.
57. The AEP-O of any one of the preceding embodiments, wherein the AEP-O comprises RAGE⁺ AT1 cells.
58. The AEP-O of any one of the preceding embodiments, wherein the AEP-O comprises cavities.
59. The AEP-O of any one of the preceding embodiments, wherein the AEP-O comprises cavities forming alveolar-like structures.
60. The AEP-O of any one of the preceding embodiments, wherein the AEP-O comprises mature and/or polarized AT1 cells within the central portion of the organoid, optionally wherein AT2 cells are intermixed with the mature and/or polarized AT1 cells.
61. The AEP-O of any one of the preceding embodiments, wherein the AEP-O comprises cavities with epithelial lining that comprises AT2 cells.
62. The AEP-O of any one of the preceding embodiments, wherein the AEP-O comprises cavities with epithelial lining that comprises AT2 cells containing lamellar bodies with the apical surface directed towards the internal lumen.
63. The AEP-O of any one of the preceding embodiments, wherein the AEP-O comprises active surfactant secretion.
64. The AEP-O of any one of the preceding embodiments, wherein the AEP-O comprises mesenchymal cells, optionally fibroblasts.
65. The AEP-O of any one of the preceding embodiments, wherein the AEP-O does not comprise mesenchymal cells, optionally fibroblasts.
66. The AEP-O of any one of the preceding embodiments, wherein the AEP-O does not comprise fibillar collagen type I and/or type II.
67. The AEP-O of any one of the preceding embodiments, wherein mesenchymal cells, optionally fibroblasts, are near and/or adjacent to the AEP-O.
68. The AEP-O of any one of the preceding embodiments, wherein the AEP-O is cultured in a container comprising a monolayer of mesenchymal cells, optionally fibroblasts.
69. The AEP-O of any one of the preceding embodiments, wherein the AEP-O comprises immune cells.
70. The AEP-O of any one of the preceding embodiments, wherein the AEP-O does not comprise immune cells.
71. The AEP-O of any one of the preceding embodiments, wherein immune cells are near and/or adjacent to the AEP-O.
72. The AEP-O of any one of the preceding embodiments, wherein the AEP-O are cultured in a container comprising a monolayer of fibroblasts comprising immune cells.
73. The AEP-O of any one of the preceding embodiments, wherein the AEP-O comprises AT1 cells expressing WNT ligands and/or PDGF ligands.
74. The AEP-O of any one of the preceding embodiments, wherein the AEP-O comprises AT2 cells that are WNT-responsive.
75. The AEP-O of any one of the preceding embodiments, wherein the mesenchymal cells are WNT-responsive, PDGFRα⁺, express HGF, express non-canonical WNT, and/or express FGF ligands.
76. The AEP-O of any one of the preceding embodiments, wherein the AEP-O comprises AEP cells expressing one or more of the AEP-enriched markers Id2, Ctnnb1, Lrp5, Lrp2, Napsa, Bex2, Hdc, and Fgfr2.
77. The AEP-O of any one of the preceding embodiments, wherein the AEP-O comprises AEP cells expressing high levels of cycle genes (pAEPs).
78. The AEP-O of any one of the preceding embodiments, wherein the AEP-O comprises AT2tr cells, optionally comprising high level expression of glutathione pathway genes and a shift towards lipid metabolism.
79. The AEP-O of any one of the preceding embodiments, wherein the AEP-O comprises mature AT2 cells expressing one or more markers selected from Sftpa1, Lys2, Sftpc and Sftpb.
80. The AEP-O of any one of the preceding embodiments, wherein the AEP-O comprises Krt8⁺ transition cells (Krt8⁺) expressing one or more markers selected from Krt8, Lgals3, Tp53, Nupr1, Ddit3, and Cldn4, or optionally one or more markers selected from Krt8, Lgals3, Tp53, and Cldn4.
81. The AEP-O of any one of the preceding embodiments, wherein the AEP-O comprises AT1 transition (AT1tr) cells expressing one or more markers selected from Hes1 and Igfbp7.
82. The AEP-O of any one of the preceding embodiments, wherein the AEP-O comprises a modification reducing or eliminating expression of Nkx2-1.
83. The AEP-O of any one of the preceding embodiments, wherein the AEP-O comprises AEPs harboring a R26R-lox-stop-lox-EYFP allele.
84. The AEP-O of any one of the preceding embodiments, wherein the AEPs are infected with AAV6.2FF-Cre.
85. The AEP-O of any one of the preceding embodiments, wherein the AEPs are from Axin2^{CreERT2-Tdt}×Rosa-EYFP× Nkx2-1^{flox/flox}×animals.
86. The AEP-O of any one of the preceding embodiments, wherein the AEPs are from Tfcp211^{CreERT2}×R26R^{EYFP}×Nkx2-1^{flox/flox}×animals.
87. The AEP-O of any one of the preceding embodiments, wherein the AEPs are from Nkx2-1 knockout animals.
88. The AEP-O of any one of the preceding embodiments, wherein the AEP-O comprises at least one alveolar-like cavity filled with debris, a pseudostratrified epithelial lining, and/or a glandular-like appearance.
89. The AEP-O of any one of the preceding embodiments, wherein the AEP-O does not comprise substantial expression of one or more foregut endoderm markers selected from Sox2, Sox9, Cdx2, Gata4, and Pdx1.
90. The AEP-O of any one of the preceding embodiments, wherein the AEP cells are from Axin2^{CreERT2-tDT} mice.
91. The AEP-O of any one of the preceding embodiments, wherein the AEP-O is embedded in an extracellular matrix.
92. The AEP-O of any one of the preceding embodiments, wherein the AEP-O is embedded in Matrigel.
93. The AEP-O of any one of the preceding embodiments, wherein the AEP-O is a model for a disease state.
94. The AEP-O of any one of the preceding embodiments, wherein the AEP-O has a genetic modification, optionally wherein the modification induces a model disease state.
95. A method comprising exposing an AEP-O of any one of the preceding embodiments to a compound.
96. The method of embodiment 95, wherein the compound is selected from a therapeutic compound, a candidate therapeutic compound, a toxin, mutagen, and/or a compound that induces a disease-like state in the AEP-O.
97. The method of embodiment 95 or 96, wherein the method comprises screening multiple compounds and/or multiple AEP-Os.
98. The method of any one of embodiments 95-97, wherein the method further comprises assessing a response of the AEP-O to exposure to compound.

### BRIEF DESCRIPTION OF THE DRAWINGS

FIGS. 1A-1M: An embodiment of AEP-derived alveolar organoids clonally expand and pattern complex, polarized alveolar-like cavities. FIG. 1A: Schematic of experimental design and overview. Live/CD31⁻/CD45-/CD326⁺ (EpCAM⁺)/TdTomato⁺ (Axin2⁺) cells (AEPs) were mixed with mouse lung fibroblasts from P28 mice and cultured for up to 35 days, followed by analysis via high content imaging.

FIG. 1B: H&E of 5 μm sections of FFPE day 35 Axin2⁺ organoids, showing cellular morphologies typical of both AT1 and AT2 cells. FIGS. 1C-1G: Whole-mount immunofluorescence time course of Axin2⁺ organoids showing expansion of SFTPC⁺ AT2 cells (red), increased differentiation into RAGE⁺ AT1 cells (green) and increased structural complexity. FIG. 1H: Imaris 3D reconstruction of day 35 Axin2⁺ organoid showing cellular arrangement/organization within mature organoids. FIG. 1I: Click-iT EdU (green) whole-mount day 25 Axin2⁺ organoids, with proliferating cells primarily on outer edges or ‘buds’ growing outward from the organoid. FIGS. 1J-1K: Electron microscopy of day 28 organoids.

FIG. 1J: Image of properly polarized AT2 cell with apical microvilli (black arrowhead) secreting surfactant (blue arrowhead) into a lumen. FIG. 1K: Image of AT2 cell with lamellar bodies (black arrowhead) adjacent to an AT1 cell (green arrowhead, right). FIGS. 1L-1M: Comparison of in vivo mouse lung (9-month C57BL/6J mouse) and in vitro day 25 Axin2⁺ organoids. [Scale bars=50 μm, except for electron microscopy (J, K) scale bars=2.5 μm]. (RAGE=Receptor for Advanced Glycation End-products [AT1 cell marker]; SFTPC=Surfactant Protein C[AT2 cell marker]; EdU=5-ethynyl-2′-deoxyuridine; FFPE=formalin-fixed, paraffin-embedded).

FIGS. 2A-2H. An embodiment of single cell composition and epithelial-mesenchymal interactions in alveolar organoids over time course of differentiation. FIG. 2A: UMAP of all cell populations combining d14, d21 and d28 AEP-derived organoid of scRNA-seq datasets. FIG. 2B: Cell population proportions at each time point with increasing proportion of epithelial cells as organoids grow. FIG. 2C: Heat map showing expression of top 10 most differentially expressed genes in each population. FIGS. 2D-2E: Ligand-receptor analysis of organoid culture demonstrating extensive mesenchymal-epithelial communication in organoids. FIG. 2F: Schematic of experimental set-up of live imaging and 3D reconstruction of live day 20 organoids generated using PDGFRaeGFP fibroblasts stained with Hoechst, with data shown in FIGS. 2F-2H. FIG. 2F: 3D reconstruction of confocal z-stacks of whole wells including transwell filter, showing the majority of GFP⁺ fibroblasts are growing on the filter; F′-F″′) Whole-mount immunofluorescence showing lack of PDGFRα⁺ cells within day 20 organoids, with scattered cells found throughout the surrounding matrigel. FIG. 2G: Whole mount IHC showing few GFP⁺ fibroblasts inside of organoids. FIG. 2H: CD45 staining of organoids; see also FIG. 121. [Scale bars=50 μm]; (PDGFRα platelet-derived growth factor receptor alpha; GFP=Green Fluorescent Protein; RAGE=Receptor for Advanced Glycation End-products [AT1 cell marker]; SFTPC=Surfactant Protein C[AT2 cell marker]); AEP=alveolar epithelial progenitor, pAEP=proliferative AEP, AT2tr=AT2 transitional cell, AT2=alveolar type 2 cell, ATItr=AT1 transitional cell, AT1=alveolar type 1 cell, Krt8=Krt8/DATP/PATS-like transitional cell, Ifn=Interferon responsive alveolar cell, pMes=proliferative mesenchymal cell, AlvFBI=alveolar fibroblast type1, AlvFB2=alveolar fibroblast type 2, SM=smooth-muscle like mesenchyme.

FIGS. 3A-3G. An embodiment of AEP-derived organoids elucidate dynamics of alveolar epithelial differentiation. FIG. 3A: scVelo RNA velocity UMAP showing differentiation dynamics (A) and pseudotime inferred from RNA velocity (A″) in AEP organoids. FIG. 3B: Slingshot trajectory analysis and pseudotime inference of AEP organoids demonstrates similar lineage relationships to RNA velocity. FIGS. 3C-3E: Lineage drivers defined by CellRank for differentiation of pAEP/AEP to AT2 cells (C), AT1 cells via AT1tr (D), and Krt8 cells (E). FIG. 3F: Heatmap showing major cell markers differentiating cell states in alveolar epithelium. FIG. 3G: Model of cellular relationships and AT1 differentiation inferred from alveolar organoids.

FIGS. 4A-4K. An embodiment of scATAC-seq analysis of AEP-derived organoid formation. FIG. 4A: UMAP of cellular populations within organoids, named according to RNA integration. FIGS. 4B-4C: Volcano plots showing differential chromatin accessibility regions between AEP and AT2 cells (B) and AT1tr and Krt8⁺ transitional cells (C). FIG. 4D: Paired heatmap of differentially accessible genomic loci in ATAC (left) and RNA expression of nearest-neighbor gene production (right) showing overview of regulators of AT1 cell differentiation (AT1 path), AT2 cell differentiation (AT2 path), and AEP state (AEP path) derived from integrated analysis. Cell populations shown along top bar, with colors the same as in (A). FIGS. 4E-4F: Pseudotime prediction of separate AT1 differentiation trajectories from AEPs to AT1 cells through AT1tr path (E) and PATS/DAPT path (F). FIG. 4G: Transcriptional activity score (TAS, negative-log p value of TF enrichment per cell type by Fisher exact test), for TFs per cell type in AT1 differentiation trajectories. FIGS. 4H-4K: Top transcriptional regulators of AEP (H), AT1tr (I), Krt8⁺ (J), and AT1 (K) cell states. Red bar=TAS, and blue bar=# of regulated genes expressed in given cell type. Network diagram shows core regulator relationship, with circle size indicating TAS, and numbers of bars showing co-regulated gene networks per cell type.

FIGS. 5A-5F. An embodiment of in vitro gene editing of AEP-derived alveolar organoids via AAV6.2FF-Cre. FIG. 5A: AAV6.2FF-Cre experimental set-up. Live/CD31⁻/CD45⁻/CD326⁺ (EpCAM⁺)/TdTomato⁺ (Axin2⁺) cells (AEPs) sorted from mice with the R26R^{EYFP} allele (Axin2^{CreERT2-tDT}; R26R^{EYFP}) were treated with AAV6.2FF-Cre and plated with wild-type fibroblasts. FIG. 5B: H&E of 5 μm sections of FFPE day 29 AAV6.2FF-Cre-treated organoids, exhibiting morphology and structural complexity similar to untreated/control organoids (FIG. 1B). FIG. 5C: Whole-well brightfield and GFP images of day 29 organoids (untreated vs. AAV6.2FF-Cre-treated) for experiment confirming MOI=1000 experimental conditions. FIG. 5D: Comparison of cells treated with an MOI of 1000, 10000, 20000. Quantification of day 32 organoids (n=3 wells per condition) showing that an MOI of 1000 causes recombination in organoids without significant effects on colony forming efficiency (CFE). FIG. 5E: Quantification (n=5 wells per condition) showing that an MOI=1000 induces significant levels of recombination without significant effects on organoid number or size. FIG. 5F: Whole-mount immunofluorescence of day 32 AAV6.2FF-Cre-treated AEP-derived organoids (same experimental set-up as FIG. 2). White box highlighting untargeted epithelial cells (YFP⁻/GFP⁻) next to a targeted (YFP⁺/GFP⁺) organoid in the same well, supporting clonal expansion of AAV6.2FF-Cre-treated cells. (ns p>0.05; *P<0.05, **P<0.01, ***P<0.001, and ****P<0.0001). Note: EYFP was stained for using anti-GFP antibodies and imaged (whole well images) using GFP filter cubes. [Scale bars=50 μm]. AAV=Adeno-Associated Virus; MOI=Multiplicity of Infection.

FIGS. 6A-6R. An embodiment of in vitro Nkx2-1 KO of AEP-derived alveolar organoids drives irreversible transition to a Krt8/PATS/DAPT-like state. FIG. 6A: AAV6.2FF-Cre experimental set-up. Live/CD31⁻/CD45⁻/CD326⁺ (EpCAM⁺)/TdTomato⁺ (Axin2⁺) cells (AEPs) sorted from Axin2^{CreERT2-tDT}; R26R-EYFP mice and Axin2^{CreERT2-tDT}; R26R-EYFP; Nkx2-1^{ﬂ/ﬂ} mice were treated with AAV6.2FF-Cre and plated with wild-type fibroblasts. FIG. 6B: Comparison of brightfield and GFP whole-well images of organoids grown from control (AAV6.2FF-Cre-treated sorted R26R^{EYFP} AEPs) and Nkx2-1 KO AEPs (AAV6.2FF-Cre-treated sorted R26R^{EYFP}; Nkx2-1^{ﬂ/ﬂ} AEPs). Control (non-GFP) organoids with normal morphology are marked with white asterisk. FIGS. 6C-6J: H&E and immunofluorescence images of R26R^{EYFP}; Nkx2-1^{ﬂ/ﬂ} AEP-derived organoids that did (F-J) or did not (C-E) undergo recombination via AAV6.2FF-Cre. (C-E) Non-recombined organoids (D) express SPC (red) and Nkx2-1 (white), but do not express the YFP lineage label (green), whereas (G) recombined organoids do not express SPC or Nkx2-1 but do express the YFP lineage label. Non-recombined (E) and recombined (H) organoids maintain epithelial identify expressing CDH1. Nkx2-1^{KO} organoids express KRT8 and many proliferate and express Ki67 expression (J-J″), as late as day 40 of culture. FIGS. 6K-6R: Integrated scRNA-sequencing datasets comparing epithelial cells from day 28 control organoids (Uninfected), AAV6.2FF-Cre-treated control organoids (AAV control), and AAV6.2FF-Cre-treated Nkx2-1 KO organoids (Nkx^{KO}). Nkx^{KO} cells cluster separately from Uninfected and AAV control cells near Krt8⁺ cells (K-L), which make up a majority of cells in the Nkx^{KO} condition (M). Marker genes for normal alveolar epithelium are lost and novel markers gained (N) in Nkx^{KO}. (O-R) Module scoring using published gene sets for AEPs (O), Krt8/PATS/DATP/ADI cells (P), lung cancer cells (Q), and foregut endoderm (R). Compare to FIG. 17 for detailed marker gene analysis.

FIGS. 7A-7N. An embodiment of genetic deletion of Nkx2-1 in vivo leads to loss of distal lung fate and acquisition of PATS/Krt8⁺ state. FIGS. 7A-7B: Experimental design of in vivo genetic ablation of Nkx2-1 in AEPs. Mouse genetic construct (A) and experimental treatment plan and schematic (B). FIGS. 7C-7N: 8-12-week Tfcp211-CreERT2; R26R^{EYFP} (C-F) and Tfcp211-CreERT2; R26R^{EYFP}; Nkx2-1^{ﬂ/ﬂ} (G-N) were treated with three doses of IP tamoxifen (50 mg/kg) and harvested at 2 to 4 weeks post-treatment; control is from 2-week timepoint. (C) Control (Tfcp211-CreERT2; R26R^{EYFP}) mice exhibited YFP induction in a subset of AT2 cells (SPC⁺ [red]/Nkx2-1⁺ [white]) with normal histological characteristics. (G-H, K-L) Nkx2-1 KO (Tfcp211-CreERT2; R26R^{EYFP}; Nkx2-1^{ﬂ/ﬂ}) mice exhibited clustered YFP⁺ proliferative clones negative for AT2 cell markers (SPC-[red]/Nkx2-1⁻ [white], H, L), with acquisition of Ki67 and Krt8 expression (I-J, M-N). Progressive clonal enlargement by 4 weeks post treatment (K) disrupts normal lung morphology, with continued growth and proliferation (all scale bars=50 μm)

FIGS. 8A-8K. An embodiment of in vitro and in vivo Nkx2-1 deletion generates a common PATS/Krt8⁺-like molecular state. FIG. 8A: Experimental design of in vivo genetic ablation of Nkx2-1 in AEPs prior to scRNAseq. FIGS. 8B-8C: Common UMAP of whole lung scRNAseq from WT and Nxk2-1^{KO} animals. (FIG. 8C) shows cell identities using LungMAP labels. FIGS. 8D-8F: Distal epithelial cell populations in WT (red) and Nkx2-1KO (blue) animals; a Nkx2-1^{KO} specific population is present which expresses markers of Krt8⁺ cells but not AT1 or AT2 cells. FIGS. 8G-8I: Overlap and label transfer of AEP-O identities to in vivo epithelial cells from FIGS. 8D-8F. FIG. 8G shows reference UMAP for comparison, reproduced from FIG. 6. (FIG. 8H) shows clustering of cells from in vivo on reference UMAP, with colors per population as in (E). Proportions of cells from WT and Nkx2-1KO are show in (FIG. 8I). FIG. 8J-8K: scATACseq of AEP-O

FIG. 9. An embodiment of a model of Nkx2-1 activity in controlling progenitor and transitional cell state. AEP can differentiate to AT1 cells via either the AT1tr or Krt8⁺ states during homeostasis, with Nkx2-1 release from AT2 genes during transition through a Krt8⁺ state. Nkx2-1 activity and expression are lowest in Krt8⁺ cells, and Nkx2-1 must re-engage chromatin to complete AT1 transition from the Krt8⁺ state. Permanent Nkx2-1 loss in AEPs causes transition to proliferative, stressed, Krt8⁺-like state characterized by unconstrained growth in vitro and in vivo. Nkx2-1 is therefore important for the progenitor activity of AEPs and supports the transition to AT1 cells by Krt8⁺ cells.

FIG. 10. An embodiment of sorting gates used to isolate Axin2-positive AT2 cells (AEPs) for use in organoids. Using the methods described herein, cells were gated away from debris based on size (1), then single cells were identified by SSC and FSC gating (2,3). Live cells were identified by Live/Dead staining (4), followed by removal of CD31- or CD45-positive cells in a dump channel (5). Epithelial cells were identified by Epcam expression (6), and TdTomato-positive epithelium were sorted into complete SAGM media and used immediately for organoids or single cell RNA sequencing (FIG. 13).

FIGS. 11A-11D. An embodiment of TUNEL+ cells largely confined to cell clumps and debris outside organoids. FIGS. 11A-11D: Whole-mount immunofluorescence and Click-iT TUNEL staining showing lack of TUNEL+ cells within mature, healthy day 25 organoids. [Scale bars=50 μm]; (TUNEL=Terminal deoxynucleotidyl transferase dUTP nick end labeling [indicator of DNA strand breaks/apoptosis]; RAGE=Receptor for Advanced Glycation End-products [AT1 cell marker]).

FIGS. 12A-12D. An embodiment of evolution and contribution of epithelial cell states per timepoint in AEP-O. FIGS. 12A-12C: UMAP projections (left) demonstrating relative detected cells for each epithelial cell state at day 14 (FIG. 12A), day 21 (FIG. 12B), and 28 (FIG. 12C). FIG. 12D: Quantification of cell population abundance at each time point.

FIGS. 13A-13I. An embodiment of localization of mesenchymal and immune cells in wells surrounding AEP-O. FIG. 13A: Generation of fibroblast stocks from control (C57BL/6J) and PDGFRrα^{eGFP} mice. FIG. 13B: Imaging of control and GFP fibroblast stocks at P2 (second passage). FIG. 13C: Whole-well scans comparing day 35 AEP-derived organoids grown from control and PDGFRα^{eGFP} fibroblasts. FIG. 13D: Quantification comparing counts and size of day 35 AEP-derived organoids grown from control and PDGFRα^{eGFP} fibroblasts. FIG. 13E: Schematic of experimental set-up of live imaging; 3D reconstruction of live day 20 organoids and PDGFRα^{eGFP} fibroblasts stained with Hoechst; (E′) 3D reconstruction of confocal z-stacks of Matrigel/organoids with the transwell filter, showing the majority of GFP⁺ fibroblasts are growing on the filter and not within organoids. FIG. 13F: Whole-mount immunofluorescence of organoids grown with PDGFRα^{eGFP} fibroblasts showing lack of PDGFRα⁺ cells within day 35 organoids. FIG. 13G: Cultures with fibroblasts on the basolateral side of the filter and AEPs on the apical side of the filter do not grow organoids (G′31-day culture), although fibroblasts do persist on the basolateral side of the filter after 31 days (G″). FIG. 13H: Confocal imaging utilizing second harmonic generation to show lack of fibrillar collagen in paraffin sections of organoids from long term culture (H″) (injured adult mouse lung section as positive control [H′]). FIG. 13I: Immunofluorescence staining showing presence of contaminating immune (CD45⁺) population (green) in fibroblast stocks (I). Whole-mount immunofluorescence of day 28 Matrigel/fibroblast mixture showing presence of contaminating immune (CD45⁺) population (green) outside organoids in transwell cultures (I′). Whole-mount immunofluorescence of day 28 organoids showing lack of immune (CD45⁺) population (green) inside organoids (I″). (ns=p>0.05) [Scale bars=50 μm].

FIGS. 14A-14B. An embodiment of the sorted AEP fraction contains both stressed and unstressed cells at baseline, but organoids arise from AEP-like AT2 by day 7 of organoid culture. FIG. 14A: scRNAseq analysis of freshly sorted AEPs. Two epithelial populations are apparent with a small mesenchymal contaminant. Right panel shows marker genes for each cell population. FIG. 14B: scATACseq of d7 AEP-O. The majority of cells at this stage are mesenchymal, with a small epithelial population which appears most similar to the pAEP state seen in late scRNAseq. Imputed gene expression from ATAC data is shown in right panel for marker gene identification. Low level expression of Krt8 markers is present, with higher level expression of AT2 genes such as Abca3 and AEP-enriched genes in the Wnt signaling pathway.

FIGS. 15A-15C. Embodiment of comparison of AEP-derived organoids to publicly available scRNA data. FIG. 15A: UMAP of AEP organoids used as basis of integration and labels. FIGS. 15B-15C: Integrated data from all three organoid datasets, labeled by cell type (FIG. 15B) or dataset of origin (FIG. 15C). Composition of each organoid dataset is shown in FIGS. 15A′-15C′.

FIG. 16. An embodiment of transition of SMAD-regulated gene expression in AT2 to AT1 transitions in AEP-O. Top row shows gene activity of SMAD target genes overlayed on scATACseq UMAP (compare to FIG. 4A). Bottom row shows gene activity Z score of SMADs in each cell population in AEP-O; BMP signaling is predicted to be higher in AT2 cells, while TGFb signaling predominates in AT1 cells.

FIGS. 17A-17P. Embodiment of Nkx2-1^{KO} organoids lack substantial protein expression of canonical endoderm markers. FIG. 17A: Time series of Nkx2-1^{KO} organoids in culture from day 12 to day 28. FIG. 17B: Immunofluorescence of paraffin section from Nkx2-1 organoid transwells showing non-recombined (Nkx2-1⁺) organoids adjacent to recombined (Nkx2-1⁻) organoids with atypical morphology. FIGS. 17C and 17J: H&E of paraffin sections of Nkx2-1Ko organoids (FIG. 17C) and E12.5 mouse embryos (FIG. 17J). FIGS. 17K′-17P′: used as positive controls for protein expression. Immunofluorescence of paraffin section from Nkx2-1^{KO} organoids (FIGS. 17D-17I) and E12.5 mouse embryos (positive controls; FIGS. 17K-17P) stained for canonical endodermal markers—Sox2 (FIGS. 17D, 17K), Sox9 (FIGS. 17E, 17L), Cdx2 (FIGS. 17F, 17M), Gata4 (FIGS. 17G, 17N), Pdx1 (FIGS. 17H, 17O), and Nkx2-1 (FIGS. 17I, 17P).

FIGS. 18A-18T. An embodiment of RNA Expression of Canonical Markers in Control (Uninfected), AAV Control, and Nkx2-1 KO AEP-derived Organoids. FIG. 18A: Overview of cell input used to generate AEP-O and subsequent timepoints for scRNAseq.

FIGS. 18B-18D: Repeated data from FIG. 6K-M; Source (FIG. 18B), cell clusters (FIG. 18C), and proportion of cell type by experimental condition (FIG. 18D) for organoids outlined in (FIG. 18A). FIGS. 18E-18T: RNA expression of common markers of alveolar epithelial cell identity.

FIGS. 19A-19D. An embodiment of efficiency of Axin2^{CreERT2} in generating biallelic knockouts in the epithelium. FIG. 19A: Experimental design to generate Nkx2-1 null AEPs using Axin2^{CreERT2}. FIG. 19B: IHC at 2 weeks post tamoxifen injection demonstrating lineage labeled AT2 cells expressing Nkx2-1 protein, suggesting incomplete knockout in the AEP lineage. FIG. 19C: Experimental design to evaluate efficiency of Nkx2-1 knockout in lineage labeled cells. FIG. 19D: Nkx2-1 expression was reduced by approximately 50% in lineage labeled cells, confirming inefficient recombination despite high dose tamoxifen via the Axin2Cre^{ERT2}.

FIGS. 20A-20L. An embodiment of Tfcp211Cre^{ERT2} functions as an epithelial-specific method to target the AEP lineage. FIG. 20A: Comparison of expression level of Axin2 and Tfcp211 in published LungMAP data shows significant epithelial enrichment. FIGS. 20B-20D: Comparison of lineage labeling in homeostatic lung using Axin2Cre^{ERT2} and Tfcp211Cre^{ERT2}. FIG. 20E: Experimental design to compare the molecular state of Tfcp211-lineage cells with Axin2Cre^{ERT2-Tdtomato} sorted AEPs. FIG. 20F: scRNAseq of sorted AEPs, reproduced from FIG. 14 for comparison. FIGS. 20G-201: UMAP project of Tfcp211^{CreERT2}×R26R^{EYFP} whole lung scRNAseq confirms Tfcp211-lineage cells comprise a subpopulation of AT2 cells in adult homeostatic lung. FIGS. 20J-20L: Comparison of molecular state of freshly sorted Axin2⁺ AT2 cells (from FIG. 20F) and Tfcp211-lineage labeled AT2 cells (from FIGS. 20G-20H). Integration of these cells leads to clustering in a single cell population, and label transfer from AEP-O scRNAseq identifies>80% of cells as in the AEP state.

FIGS. 21A-21D. An embodiment of Tfcp211-lineage organoids form complex organoids in AEP-O culture conditions. FIG. 21A: Experimental design for generation of Tfcp211-lineage organoids. FIG. 21B: Whole mount image showing virtually all organoids in culture derive from Tfcp211-lineage labeled EYFP+ cells. FIGS. 21C-21D: Tfcp211-derived organoids form complex organoids which develop complex cellular differentiation and 3D organization indistinguishable from Axin2-lineage organoids.

### DETAILED DESCRIPTION

In embodiments disclosed herein, we refined and standardized the culture conditions and inputs of co-culture of murine AT2 cells and alveolar fibroblasts. Recent data demonstrates that Wnt-responsive AT2 cells, also called alveolar epithelial progenitors (AEPs), harbor extensive progenitor capacity. Following injury, AEPs expand rapidly, differentiate into new AT1 and AT2 cells, and repair regions of alveolar injury following epithelial loss or infectious stress. Herein, we describe the high dimensional characterization of these organoids across multiple timepoints. We find that AEP-derived organoids, or AEP-O, develop from clonal expansion of single progenitor cells, undergo progressive cellular differentiation and spontaneous cavity formation in vitro, giving rise to complex alveolar-like structures with properly polarized epithelial cells, recapitulating key aspects of the alveolar regenerative process in a flexible in vitro assay. Using multistage single cell transcriptomics and epigenomics, we defined the organoid cellular milieu, identified separable progenitor, AT2, AT1, and transitional states in organoids. We validated these states based on published in vivo scRNAseq and derived cellular trajectories and lineage relationships from a known progenitor root state. Comparative transcriptional regulatory network (TRN) analysis along these trajectories identified several known and novel regulators of alveolar epithelial biology and highlighted a novel role for the lineage transcription factor Nkx2-1 in homeostatic and stressed transitional lineages of the adult alveolus. Genetic ablation of Nkx2-1 in AEPs in vitro and in vivo caused irreversible acquisition of the stressed transitional state, with uncontrolled proliferative growth of these cells and disruption of organoid morphology in vitro and alveolar structure in vivo. These findings highlight the utility of the AEP-O assay as a high-fidelity model of in vivo alveolar biology and implicate Nkx2-1 in AEPs as a central regulator of alveolar epithelial progenitors.

### Alveolar Epithelial Progenitor-Derived Lung Organoids Recapitulate Key Cellular and Morphological Aspects of Alveolar Regeneration.

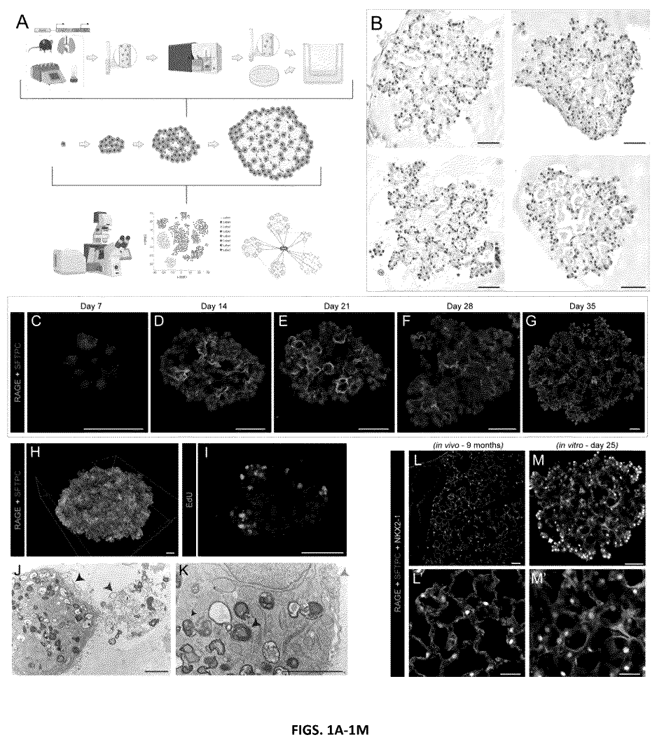

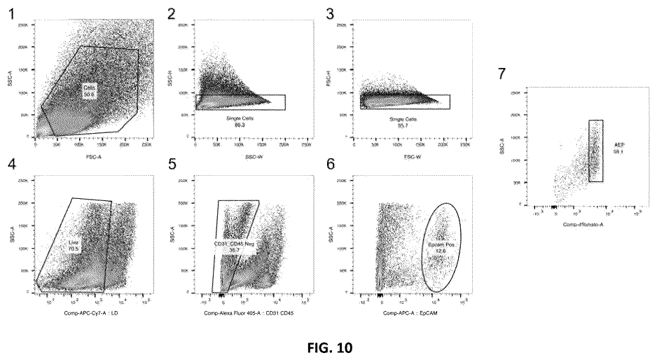

To optimize aspects of the AEP-O culture, we undertook extensive reagent testing. In some embodiments, the primary components of organoid co-culture assays are 1) epithelial cells; 2) supportive cells, if any; 3) matrix for three-dimensional suspension and growth; 4) media and media additives; and 5) growth surface (e.g., transwell filter). We tested each of these components iteratively. As previously reported, AEPs form more and larger organoids than unselected AT2 cells, so in some embodiments, we used FACS-sorted AEPs (FIG. 10). In some embodiments, the AEPs are derived from Axin2^{CreERT2-Tdt} mice as the epithelial starting fraction. In some embodiments, we used supportive mesenchymal cells. In some embodiments, we used large, consistent preparations of primary lung fibroblasts. In some embodiments, the fibroblasts are obtained by selective adhesion from P28 wild type C57BL/6 mice at passage 3-4. We then lot tested 4 available commercial matrices, all with composition similar to Matrigel (Coming), and found that the best lots of Matrigel support organoid growth with 2-3-fold increased CFE compared to other Matrigel lots and competing products; we therefore purchased an entire lot of Matrigel and all data herein uses this standardized reagent. In some embodiments, Matrigel is used. In some embodiments, other matrices are contemplated. In some embodiments, we cultured 5,000 sorted AEPs and 50,000 lung fibroblasts in each well on a transwell filter in a 1:1 ratio of Matrigel and small airway growth media ((SAGM) (Lonza)) with 5% FBS and limited additives); we took this approach minimize exogenous signaling modulators in the media and allow evaluation of the supportive capacity of the mesenchymal fraction. In some embodiments, about 1,000, 2,000, 3,000, 4,000, 5,000, 6,000, 7,000, 8,000, 9,000, or 10,000, or a range defined by any two of the preceding values, of sorted AEPs and about 10K, 20K, 30K, 40K, 50K, 60, 70K, 80K, 90K, or 100K, or a range defined by any two of the preceding values, mesenchymal cells, e.g., lung fibroblasts, are added to each well on a transwell filter. In some embodiments, in a ratio of AEPs to mesenchymal cells of 2:1, 1:1, 1:2, 1:5, 1:10, 1:15, 1:20, 1:50, or a range defined by any two of the preceding values. In some embodiments, they are added in Matrigel and media (optionally small airway growth media (SAGM) with about 1-10%, optionally about 5%, FBS and optionally with limited additives as disclosed herein, in a ratio of about 10:1, 5:1, 3:1, 2:1, 1:1, 1:2, 1:3, 1:5, or 1:10, or a range defined by any two of the preceding values. To evaluate the growth of these organoids more fully, we adapted methods from iPSC culture for whole mount immunohistochemistry of organoids and performed a time series evaluation using high content confocal imaging with paired single cell sequencing (FIG. 1A).

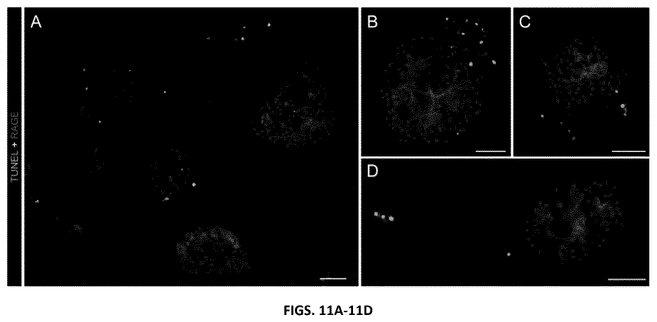

In some embodiments, AEP-O grow clonally from a single AEP in definable stages. First, AEPs expand into small clusters of SFTPC⁺ cells during the first week of culture (FIG. 1C). By day 14 of culture, differentiation of RAGE⁺ AT1 cells was observed within the central portion of the organoids (FIG. 1D), consistent with previous reports from murine organoids. During the third week of culture, these developing AT1 cells begin to elongate and polarize (FIG. 1E), and by day 14 of culture cavities are present within the organoids (FIG. 1B, F). These cavities mature into an intricate network of alveolar-like structures during the 4^{th} and 5^{th} weeks of differentiation (FIG. 1F-G), with AT1 cells intermixed with AT2 cells within the central portion of the organoid (FIGS. 1H, 1M), with minimal apoptosis detectable by TUNEL staining in AEP-O during cavity formation (FIGS. 11A-11D). Continued proliferation evident by EdU incorporation around the external aspect of mature organoids (FIG. 1I). Electron microscopy demonstrated that the epithelial lining of these cavities also includes AT2 cells containing lamellar bodies with the apical surface directed towards the internal lumen and evidence of active surfactant secretion (FIG. 1J-K). These features of the mature cavities within AEP-O bear a striking similarity to the epithelial structure of mature murine alveoli (FIG. 1L-M) compared to other reported lung organoids. In some embodiments, the AEP-O comprise AEP cells, AT1 cells, and AT2 cells, optionally wherein the AEP, AT1 and AT2 cells express markers disclosed herein. In some embodiments, the AEP-O comprises RAGE⁺ AT1 cells. In some embodiments, the AEP-O comprise cavities, optionally forming alveolar-like structures. In some embodiments, the AEP-O comprises mature and/or polarized AT1 cells within the central portion of the organoid, optionally wherein AT2 cells are intermixed with the mature and/or polarized AT1 cells. In some embodiments, the AEP-O comprise cavities with epithelial lining that comprises AT2 cells, optionally containing lamellar bodies with the apical surface directed towards the internal lumen. In some embodiments, the AEP-O comprises active surfactant secretion.

AEP-O Maturation is Driven by Mesenchymal Paracrine Signaling without Direct Mechanical Contribution of Mesenchymal Cells.

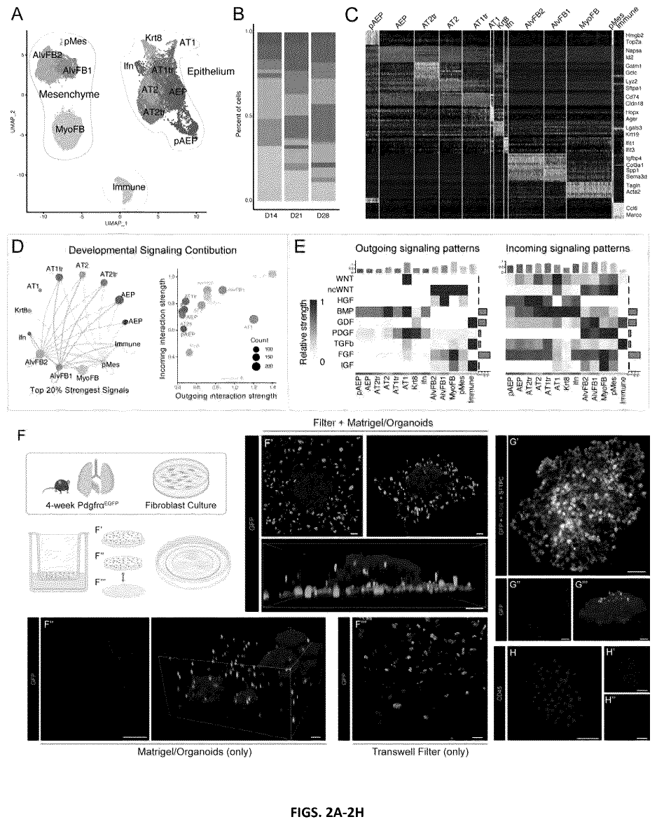

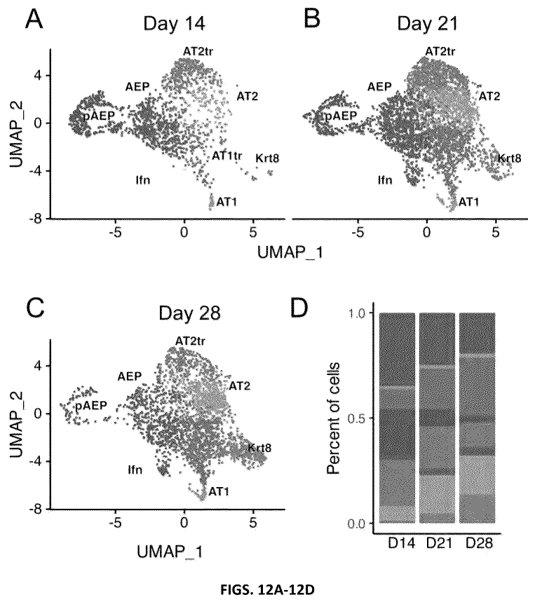

To better characterize the progressive cellular maturation occurring during paired cell differentiation and cavity formation in AEP-O, we performed single cell RNA sequencing at 14, 21, and 28 days after culture initiation. We identified clear epithelial and mesenchymal fractions, as well as an unexpected immune fraction (FIG. 2A-C). Epithelial contribution increases from d14 to d28, with progressive maturation of the epithelium and increase in relative proportion of alveolar type 1 cells (FIG. 2B, FIGS. 12A-12D).

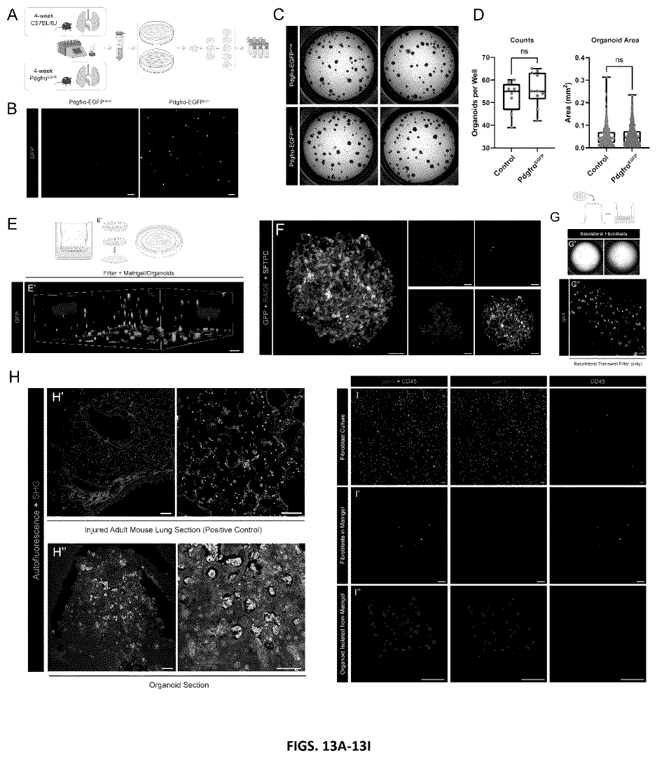

Within the mesenchyme, we noted two major PDGFRα⁺ populations, corresponding to murine alveolar fibroblasts and myofibroblasts in previously annotated LungMAP datasets. To localize these mesenchymal cells in complex culture, we obtained fibroblasts from d28 PDGFRα^{eGFP} mouse lungs via selective adhesion (FIGS. 13A-13B) and confirmed that these cells were largely GFP positive prior to addition to organoids (FIG. 13B). Use of these PDGFRα^{eGFP+} fibroblasts did not affect organoid growth (FIGS. 13C-13D). We used these cells to support AEP-O growth, and performed live imaging of GFP fluorescence to localize GFP⁺ cells in whole wells, isolated Matrigel plugs, and transwell filters (FIG. 2F-F″′). This was followed by whole-mount immunofluorescence of isolated organoids (FIG. 2G). PDGFRα^{eGFP+} fibroblasts localize predominantly in two locations—with the minority of cells surrounding the epithelial organoids suspended in matrigel, and the majority growing on the transwell filter in a monolayer (FIGS. 2F-2H, FIGS. 13E-13F). Few PDGFRα^{eGFP+} cells were detected within organoids (FIG. 2F-2G) and no clear deposition of fibrillar collagen (types I and II) was seen within organoids (FIG. 13H), suggesting that the morphological maturation and complex structural organization of AEP-O did not require direct mesenchymal cell localization within the organoid itself. CD45 staining showed immune cells were scattered throughout the fibroblast stocks and within the Matrigel, but never found in large clusters or within organoids (FIG. 13I). In some embodiments, the AEP-O comprises mesenchymal cells, e.g., fibroblasts. In some embodiments, the AEP-O does not comprise mesenchymal cells, e.g., fibroblasts. In some embodiments, the AEP-O does not comprise fibillar collagen type I and/or type II. In some embodiments, mesenchymal cells, e.g., fibroblasts, are near and/or adjacent to the AEP-O. In some embodiments, the AEP-O is cultured in a container comprising a monolayer of mesenchymal cells, e.g., fibroblasts. In some embodiments, the AEP-O comprises immune cells. In some embodiments, the AEP-O does not comprise immune cells. In some embodiments, immune cells are near and/or adjacent to the AEP-O. In some embodiments, the AEP-O are cultured in a container comprising a monolayer of fibroblasts comprising immune cells.

Ligand-receptor analysis suggested extensive signaling between epithelial and mesenchymal cells within AEP-O (FIG. 2D-E). Major signal producers in AEP-O included AT1 cells, matrix fibroblasts and myofibroblasts. AT1 cells expressed extensive WNT ligands with predicted receptivity in both WNT-responsive AT2 cells and multiple mesenchymal populations. They also had high level expression of AT1 also produced PDGF ligands predicted to signal to the PDGFRα⁺ mesenchyme. Mesenchymal cells expressed HGF, non-canonical WNT, and FGF ligands, consistent with published data describing roles of these pathways in alveolar regeneration. Together, these data suggest that the AEP-O signaling milieu recapitulates key aspects of the in vivo regenerative niche, and that the mesenchymal cells provide primarily a supportive paracrine signaling niche important for alveolar cavity formation. To directly evaluate the importance of mesenchymal signaling support, we cultured of PDGFRα^{eGFP+} cells on the basal side of the transwell filter (FIG. 13G). Absence of fibroblasts in the Matrigel plug led to complete loss of organoid formation in AEP-O (FIG. 13G), confirming the importance of a nearby supportive mesenchymal niche signaling in the establishment and maturation of AEP-O. In some embodiments, AT1 cells of the AEP-O express WNT ligands and/or PDGF ligands. In some embodiments, AT2 cells of the AEP-O are WNT-responsive. In some embodiments, mesenchymal cells cultured with the AEP-O are WNT-responsive, PDGFRα⁺, express HGF, express non-canonical WNT, and/or express FGF ligands.

scRNAseq Defines Separable Epithelial Maturation Trajectories of AEPs Toward AT1 and AT2 Cells within Alveolar Organoids.

We next turned our attention to epithelial differentiation in AEP-O. Integrated analysis of data from d14, 21, and 28 identified eight separable epithelial cell states via graph-based clustering in Seurat (FIGS. 2A-C, FIGS. 12A-12D). Concordant with our whole mount IHC results, we detected both AT1 and AT2 cell states within organoids. We noted a clear AEP state defined by expression of the AEP-enriched markers Id2, Ctnnb1, Lrp5, Lrp2, Napsa, Bex2, Hdc, and Fgfr2. Cells bearing this AEP signature were also found in a second state partially defined by high level expression of cell cycle genes. We called these cells AEPs and proliferative AEPs (pAEPs) respectively (FIG. 2A). As expected, the proliferative state is readily detectable in d14 cultures and decreases by d28 (FIGS. 12A-12D). Consistent with these findings and previous reports, EdU staining of organoids demonstrates a proliferative Sftpc⁺ positive population (FIG. 1I). In some embodiments, the AEP-O comprises AEP cells expressing one or more of the AEP-enriched markers Id2, Ctnnb1, Lrp5, Lrp2, Napsa, Bex2, Hdc, and Fgfr2. In some embodiments, the AEP-O comprises AEP cells expressing high levels of cycle genes (pAEPs).

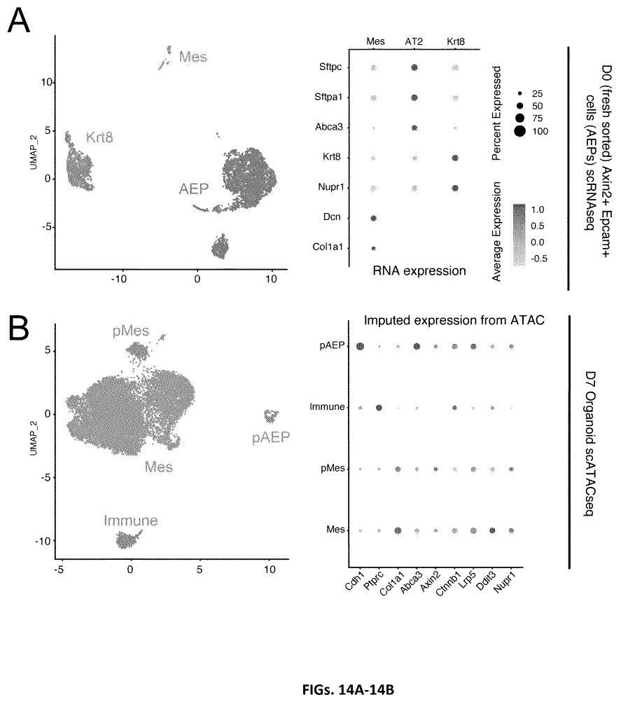

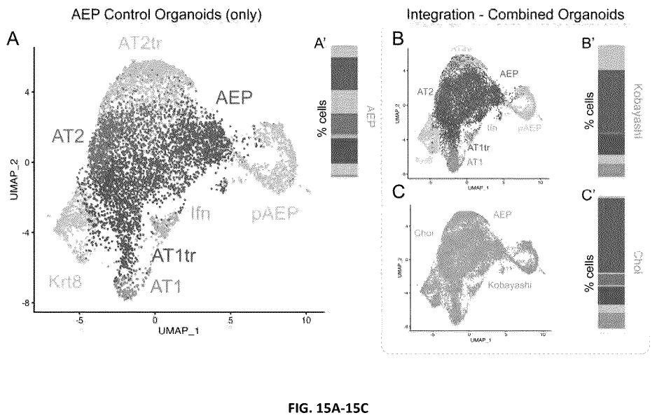

We then examined the initiating cells of these organoids in more detail. The starting epithelial fraction of AEP-O is comprised of sorted AEPs (as described in and shown in FIG. 10). scRNAseq of AEPs immediately after sorting showed two distinct fractions of epithelial cells and a tiny fraction of contaminating mesenchyme (FIG. 14A). The larger epithelial fraction corresponded to AT2 cells bearing the AEP signature and a second smaller state contained cells in the Krt8+ stressed transitional state (also referred to herein as “Krt8⁺/PATS/DATP/ADI” cells) (FIG. 14A), suggesting that process of digestion and sorting of Axin2+ AT2 cells causes cell stress. This signature is consistent with published reports in other organ systems, with prior data specifically suggesting a clear impact on cellular oxygenation and metabolism during tissue digestion for FACS. Evaluation of scATACseq analysis at d7 of AEP-O culture (FIG. 14B) demonstrated that the relatively small fraction of epithelium at this time point is a single population most similar to pAEPs, suggesting that the AT2/AEP fraction of the sorted cells seen by scRNAseq following FACS is the likely initiating epithelial cell in AEP-O, though a contribution of Krt8+ cells which then revert to the AEP state cannot be excluded by our data.

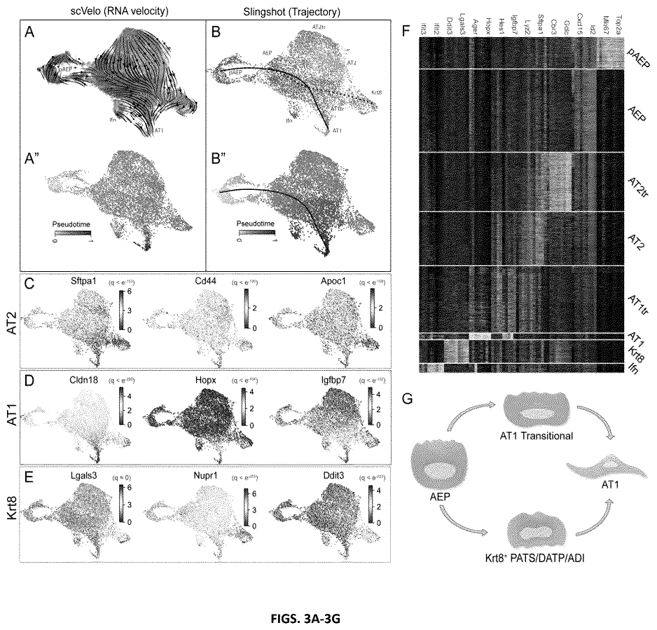

To dissect pathways of differentiation from the AEP state, we compared the lineage predictions generated by both trajectory analysis and RNA velocity (FIG. 3A-B). We detected differentiation of AEPs toward AT2 cells through an AT2 transitional intermediate state (AT2tr) (FIG. 3C). The AT2tr state is defined by high level expression of glutathione pathway genes and shift towards lipid metabolism, with the AT2 state expressing high levels of mature AT2 markers including Sftpa1 and Lys2 in addition to Sftpc and Sftpb, which are expressed broadly within multiple differentiating AEP states (FIG. 2C, 3F). In some embodiments, the AEP-O comprises AT2tr cells, optionally comprising high level expression of glutathione pathway genes and a shift towards lipid metabolism. In some embodiments, the AEP-O comprises mature AT2 cells expressing one or more markers selected from Sftpa1, Lys2, Sftpc and Sftpb. These predictions correspond to the lineage trajectories implied by the progression of organoid composition shown in the time course data underlying the combined set (FIGS. 12A-12D).

We next examined AT1 differentiation in AEP-O. Significant recent attention has focused on differentiation of AT2 progenitor cells to AT1 cells, with multiple reports describing a distinct transitional state (variously called PATS, DATP, AD1, or Krt8⁺ cells, also referred to herein as “Krt8⁺/PATS/DATP/ADI” cells) marked by high level expression of cell stress markers including Krt8, Lgals3, Tp53, and Cldn4, as well as Nupr1, Ddit3. This stressed cell state, which we will refer to as the Krt8⁺ transition cell (Krt8⁺) is readily detectable in AEP-O, though we noted that both trajectory analysis and RNA velocity suggested two trajectories toward AT1 cells; one through this Krt8⁺ transitional state, and another through a second, less stressed state we denoted AT1 transition (AT1tr). RNA markers of the AT1tr state included Hes1 and Igfbp7, and RNA velocity analysis showed decreasing AT2 gene expression and intermediate expression of AT1 markers within this population (FIG. 3D-F). Label transfer and integration with published organoid data sets showed AT1tr and Krt8⁺ cell states were detectable in published data (FIGS. 14A-C). These findings suggested the model that AEPs could differentiate toward AT1 cells through either the Krt8⁺ state or the AT1tr state (FIG. 3G), rather than through an obligate intermediate state. However, given the challenges of cell state predictions and transitions based on solely on RNA transcriptome, we proceeded to evaluate epigenomic state and chromatin topography of the cells comprising AEP-O via scATACseq. In some embodiments, the AEP-O comprises Krt8⁺ transition cells (Krt8⁺) expressing one or more markers selected from Krt8, Lgals3, Tp53, Nupr1, Ddit3, and Cldn4, or optionally expressing one or more markers selected from Krt8, Lgals3, Tp53, and Cldn4. In some embodiments, the AEP-O comprises AT1 transition (AT1tr) cells expressing one or more markers selected from Hes1 and Igfbp7.

### AEP Chromatin State is Defined by a Progenitor-Enriched Transcriptional Regulator Network.

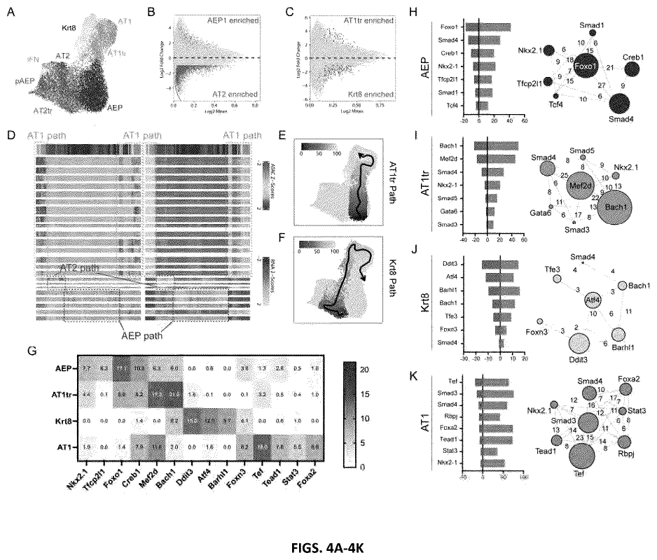

We performed scATAC sequencing at d14, d21, and d28 in AEP-O, and performed unbiased cell clustering of epigenomic states using ArchR. Remarkably, we detected the same number of epithelial cell states by either scRNAseq or scATAC using default parameters, suggesting appropriate clustering in both assays (FIG. 4A). Concordant with published bulk ATACseq data, AEP and AT2 are distinct at the epigenomic level (FIG. 4B); AT1tr and Krt8⁺ also showed differential chromatin accessibility, with significantly distinct regions of open chromatin (FIG. 4C). Integrated analysis combining both scRNA and scATACseq identified separable clusters of regulated genes within AEPs, for AT2 differentiation, and for AT1 differentiation (FIG. 4D). scATACseq-based pseudotime inference confirmed the presence of two separate differentiation trajectories from AEPs to AT1 cells, one passing through the Krt8⁺ state and the second passing through the AT1tr state (FIG. 4E-F), concordant with RNA analysis. Comparison of expression and chromatin accessibility using integrated analysis suggested overlapping gene sets shared by Krt8⁺ and AT1tr cells, but also substantive differences (FIG. 4D), implying independent regulatory inputs to these two states.

AEP to AT1 Differentiation Occurs Through Transitional with Partially Overlapping TRNs.

To better define the Krt8⁺ and AT1tr states, we turned to transcriptional regulatory network (TRN) inference. Transcriptional regulatory network inference models describe interactions between transcription factors and their gene targets within specific cell types, providing predictions regarding both the driving TFs and the regulated gene sets in a particular cell type. Combination of RNA expression and chromatin accessibility can improve TRN predictions by reducing both false positive and false negative regulatory predictions; application to single cell techniques have extended the power of these approaches to estimate TF regulators of individual cell states. Therefore, we performed TRN inference comparing regulatory networks of various cell states within AEP-O to identify differential TF activity and predict regulators of state transitions (FIG. 4G-K). Focusing on the core TFs specific to each TRN, we found that AEP regulators included Nkx2-1 and Tfcp211, both enriched in expression in bulk RNAseq from AEPs, as well as TFs modulating Wnt (Tcf4) and BMP (Smad¼) activity concordant with known AT2 progenitor signaling response (FIG. 4H). Within AT1 cells, we identified Nkx2-1, Gata6, and Foxa2, all well-known modulators of AT1 gene expression, and signaling response from the AT1-associated Yap/Taz (Tead1), Notch (Rbpj), and Tgfβ (Smad¾) signaling pathways (FIG. 4K). These results provided evidence that the TRN inference had successfully identified known regulators of the AEP and AT1 cell states, supporting the notion that TRN inference could distinguish factors driving AT1 differentiation.

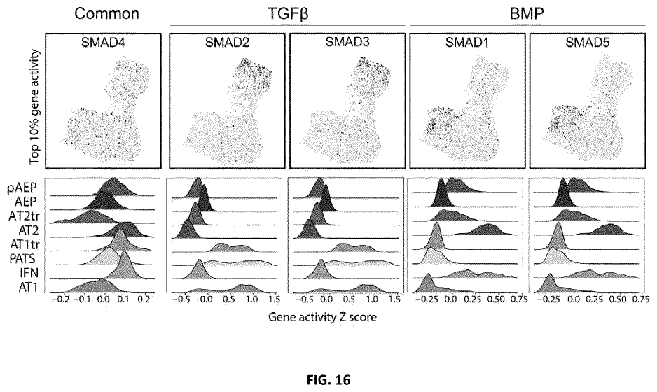

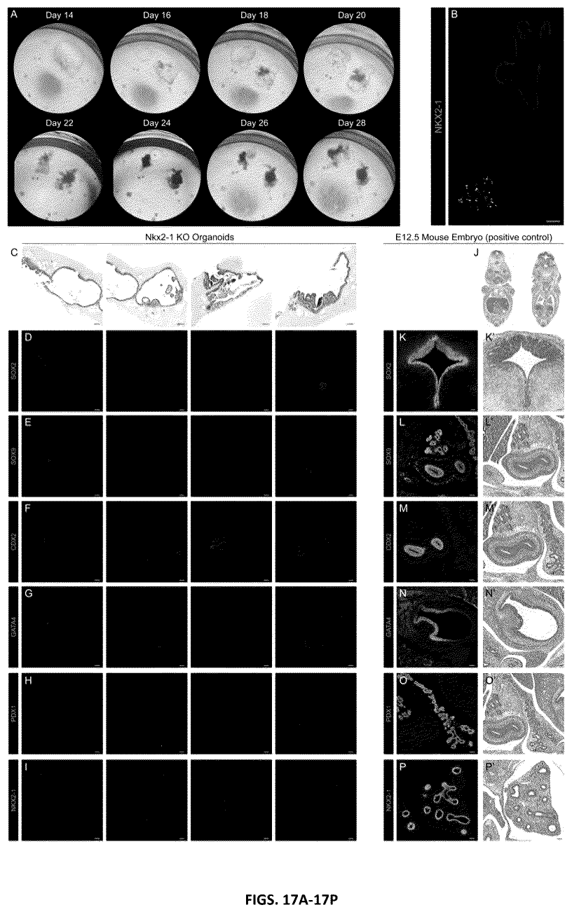

We therefore focused our attention on differential regulators of transitional cells. We calculated transcription factor activity scores based on enrichment in predicted cell type. Both states showed overlap with AT1 enriched TFs. The TRN predictions were otherwise distinct, suggesting differential inputs to the two transitional states. The top regulators in Krt8⁺ cells are Atf4 and its target Ddit3, the gene encoding C/EBP homologous protein (CHOP) (FIG. 4J); together these factors are activated by the multiple inputs of the integrated stress response (ISR), with CHOP implicated in regulation of checkpoints in apoptosis vs cellular differentiation in other systems. The ISR is activated in alveolar epithelium following ventilator-induced lung injury, and ISR activation contributes to lung fibrosis; Atf4 and CHOP activation underlies Krt8⁺ cell accumulation in fibrosis. The AT1tr TRN showed multiple distinct factors which are shown for comparison (FIG. 4K and FIG. 16); given the overlap of these factors with both AT2 and AT1 cells, testable regulators of the AT1tr state were not clearly identifiable in our dataset.

### Loss of Nkx2-1 Activity Defines the Krt8⁺ Transitional Cell TRN.

Notably, we identified a surprising absence of predicted Nkx2-1 activity in Krt8⁺ cells; TRNs from all other epithelial cell states in AEP-O included Nkx2-1. Recent epigenomic profiling of the activity of Nkx2-1 during AT2 to AT1 transitions demonstrated Nkx2-1 occupancy at different genomic regions in each cell state, suggesting a role for Nkx2-1 dis-engagement and re-engagement in the genome during differentiation. Nkx2-1 expression is lowest in stressed transitional epithelial cells at the time when Krt8 expression is highest during in vivo lung regeneration. Together, these observations supported the hypothesis that lack of Nkx2-1 activity promotes transition to the stressed Krt8⁺ transitional state.

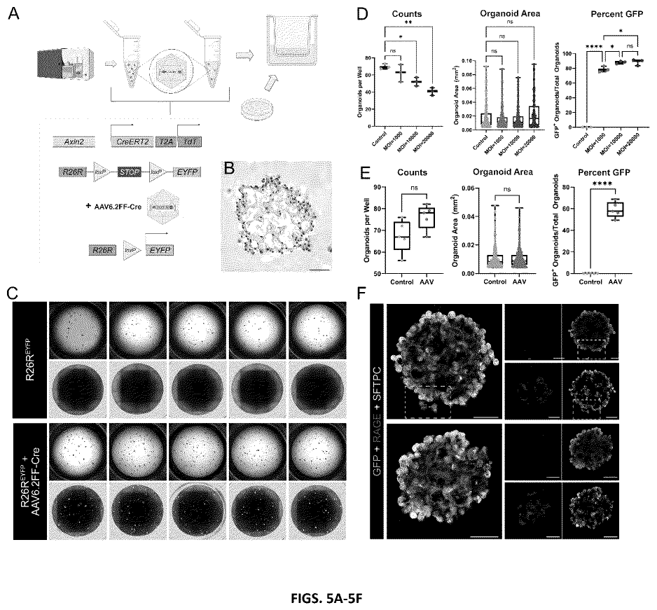

To validate this observation, we developed an approach to genetically manipulate AEPs during development of AEP-O (FIG. 5). Using an AAV6.2FF-Cre, which has recently been described as a high-fidelity reagent for genetic manipulation of AT2 cells in vivo, we infect AEPs harboring a R26R-lox-stop-lox-EYFP allele (from R26R^{EYFP} mice) immediately after FACS sorting (FIG. 5A). AAV6.2FF-Cre efficiently targeted AEPs, which produced morphologically complex organoids (FIG. 5B) expressing the EYFP lineage label (FIG. 5C). Titration experiments indicated that infection at a multiplicity of infection (MOI) of 1000 was sufficient to induce significant recombination and label the majority of AEP-O (FIG. 5D); higher MOI mildly increased targeting, but at the expense of reduction in colony formation. Multiple biological replicates confirmed that MOI=1000 led to targeting of approximately 60% of organoids with no change in colony formation efficiency or size of organoids (FIG. 5E); whole mount IHC confirmed EYFP expression with no reduction in internal complexity of AEP-O (FIG. 5F). These results indicated that AAV6.2FF-Cre was capable of efficiently targeting AEPs in vitro for genetic manipulation without perturbing the AEP-O system, and confirmed the clonal nature of AEP-derived organoids.

Nkx2-1 deficient AEPs Transition to the Krt8+ Stressed Transitional Cell State.

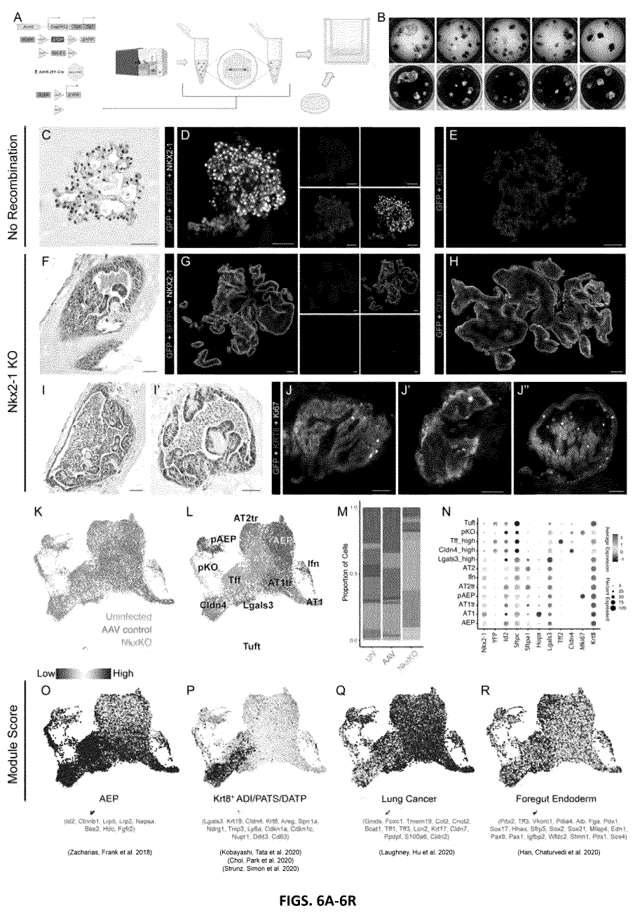

To test the hypothesis that Nkx2-1 deficiency contributed to the Krt8⁺ state transition, we applied AAV6.2FF-Cre to AEPs from Axin2^{CreERT2-Tdt}×R26R^{EYFP}×Nkx2-1^{flox/flox} animals to generate Nkx2-1 knockout AEPs which were used to initiate organoid formation (FIG. 6A). Morphology in EFYP⁺ (Nkx2-1^{KO}) organoids was noticeably different from EYFP-organoids in the same well, and EFYP⁺ organoids were substantially larger by day 28 of culture (FIG. 6B). Because AAV6.2FF-Cre only targeted ~60% of organoids per well, we were able to directly compare EFYP⁺ and EFYP⁻ organoids grown in the same well to directly assess the impact of Nkx2-1 knockout in AEP-O. As expected, we noted robust Nkx2-1 protein expression in EFYP⁻ organoids (FIG. 6C-E), and complete loss of Nkx2-1 protein expression in EFYP⁺ organoids. Nkx2-1^{KO} AEPs lost expression of AT2 markers, including Sftpc, with associated increased expression of E-cadherin (Cdh1) and change in cell shape and organoid morphology (FIG. 6F-H). Diverse morphological types were visible in Nkx2-1^{KO}AEP-O, with loss of alveolar-like cavities, prominence of one or a small number of large cavities full of debris, and pseudostratified epithelial lining with some organoids exhibiting a glandular appearance (FIG. 6F,I). These structures were reminiscent of other endoderm-derived organs, including the esophagus, stomach, and intestine, and Nkx2-1 knockout in distal lung has been associated with expression of foregut endoderm genes, so we examined protein expression of a large panel of proximal lung and foregut endoderm markers including Sox2, Sox9, Cdx2, Gata4, and Pdx1. No substantial expression of any of these markers was detectable, suggesting Nkx2-1^{KO} AEP-O epithelium did not adopt foregut endoderm fates from proximal lung or GI organs (FIGS. 16A-16P). In some embodiments, the AEP-O comprises at least one alveolar-like cavity filled with debris, a pseudostratrified epithelial lining, and/or a glandular-like appearance. In some embodiments, the AEP-O does not comprise substantial expression of one or more foregut endoderm markers selected from Sox2, Sox9, Cdx2, Gata4, and Pdx1.

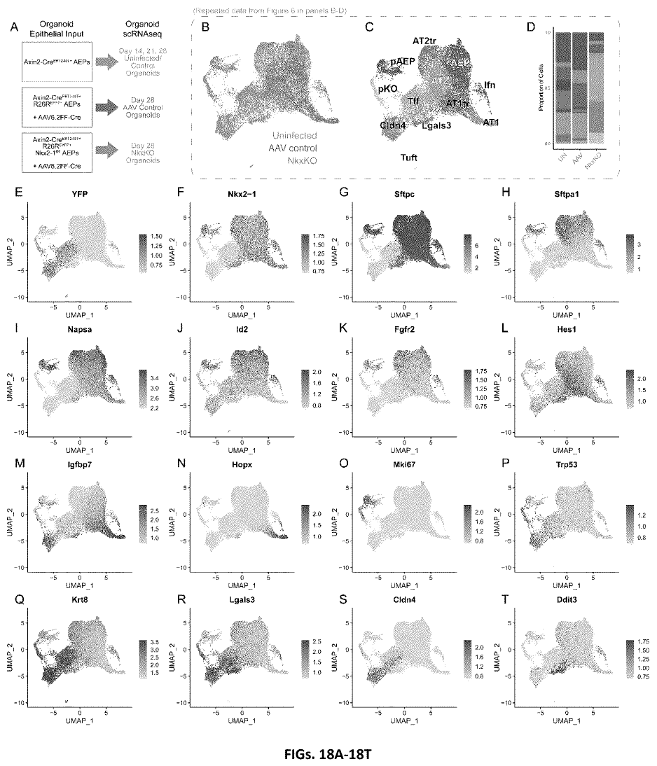

To clarify Nkx2-1^{KO} AEP-O composition, we turned to unbiased profiling. We performed scRNAseq in Nkx2-1 KO AEP-O at 28d of culture and compared the expression profile of Nkx2-1^{KO} cells with Nkx2-1 expressing cells in both our WT organoid scRNAseq time series. We added a control condition of AAV6.2FF-Cre treatment in AEP-O from Axin2^{CreERT2-Tdt}×R26R^{EYFP} (as in FIG. 5), to rule out any AAV6.2FF-Cre-specific effects. We then integrated scRNAseq expression data from Nkx2-1^{KO} and Nkx2-1^{+/+} AEP-O and compared gene expression profiles (FIGS. 6K-6R, FIG. 18). Consistent with IHC, Nkx2-1 and EYFP RNA expression are mutually exclusive. Nkx2-1 was undetectable in EYFP⁺ epithelial cells at the RNA level (FIG. 6N). EYFP⁺ Nkx2-1^{KO} cells form multiple distinct clusters separated from Nkx2-1 expressing cells in both EYFP-cells of the same wells and the two control libraries (wild-type/uninfected and AAV control) (FIG. 6K). Nkx2-1^{KO} cells clustered near WT Krt8⁺ transitional epithelial cells than other control clusters (FIG. 6L) and expressed high levels of markers of Krt8⁺/PATS/DATP/ADI state including Cldn4 and Tff2, while expressing intermediate levels of Lgals3 (FIGS. 6N, 18Q-18T). A distinct proliferative cluster is present by scRNAseq among Cldn4-high Nkx2-1^{KO} cells (FIG. 6K). Ki67 expression in Krt8⁺ Nkx2-1^{KO} AEP-O confirms ongoing proliferation at d40 of culture despite their large size (FIG. 6J).

Given the similarities of Nkx2-1^{KO} epithelial cells to the Krt8⁺ state in Nkx2-1 WT AEP-O, we used Seurat module scoring to compare Nkx2-1KO and WT cells. Cells in Nkx2-1^{KO} AEP-O lost AEP-associated gene expression (FIG. 6O) while activating cell stress markers associated Krt8⁺ cells in WT AEP-O. Cells from Nkx2-1^{KO} AEP-O were highly enriched for gene sets associated with human lung adenocarcinoma, concordant with previous findings implicating Nkx2-1 loss in the pathogenesis of lung cancer. While we did not detect non-lung foregut endoderm markers by IHC, Nkx2-1^{KO} epithelial cells did express low levels of non-lung endodermal genes at the RNA level, consistent with loss of instructive activity of Nkx2-1 in constraining lung epithelial cell fate. Taken together, these data supported the conclusion that Nkx2-1 loss in AEPs led to acquisition of the Krt8⁺/PATS/DATP/ADI cell state, validating the prediction of the Krt8⁺ cell state TRN analysis.

### Deletion of Nkx2-1 in AEPs In Vivo Caused Rapid, Spontaneous Conversion to a Proliferative Krt8⁺ Transitional State.

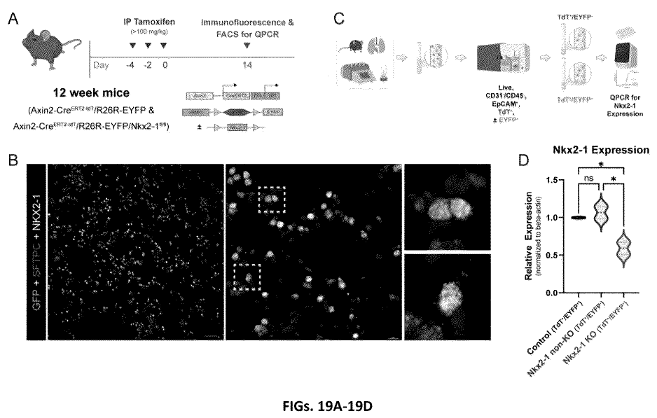

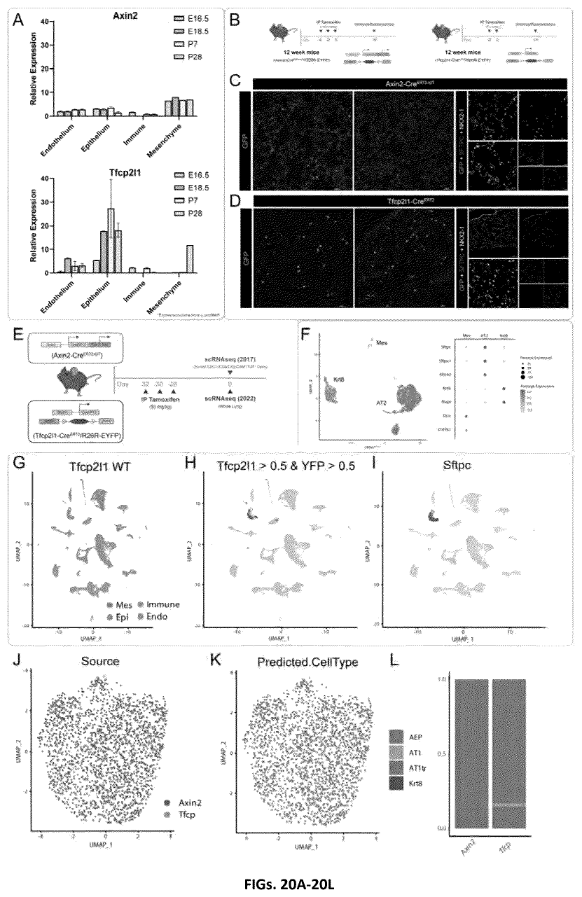

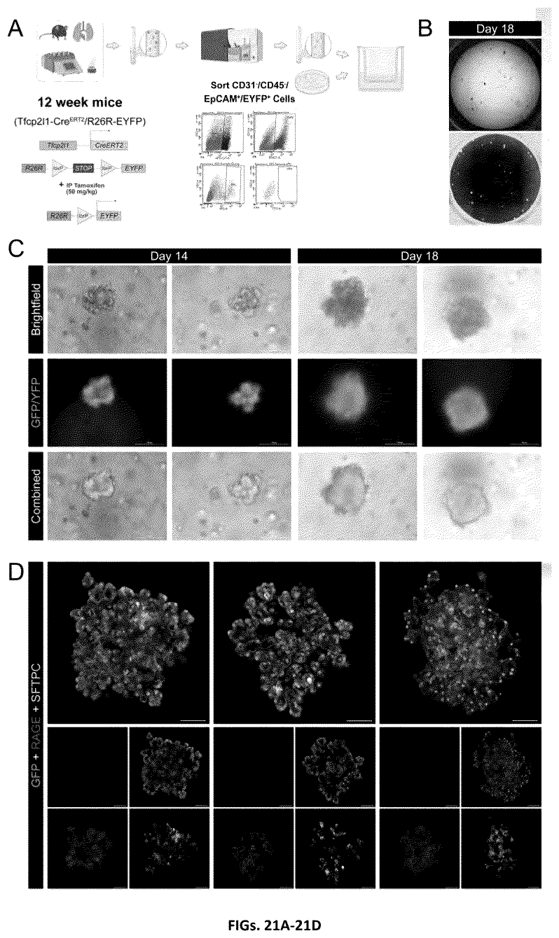

To further evaluate the hypothesis that Nkx2-1 loss is sufficient to cause acquisition of the Krt8⁺/PATS/DATP/ADI cell state in AEPs, we performed in vivo lineage tracing of Nkx2-1^{KO} AEPs in adult mice at homeostasis. While Axin2^{CreERT2} functions as an effective lineage tracing reagent for lung epithelial cells after high dose tamoxifen treatment, the relative CreERT2 recombination inefficiency with this line full deletion of floxed alleles in lineage labeled cells in adult lung epithelium at high doses of tamoxifen (FIGS. 19A-19D). We therefore turned our attention to the recently reported epithelial-specific Tfcp211^{CreERT2}mouse line. We performed lineage tracing of Tfcp211^{CreERT2}×R26R^{EYFP} animals and detected lineage label in solitary AT2 cells (SFTPC⁺/NKX2-1⁺) scattered throughout individual alveoli after administration of tamoxifen with similar distribution to Axin2^{CreERT2}(FIG. 19D). We then performed scRNAseq on Tfcp211^{CreERT2}×R26R^{EYFP}; EYFP labeled cells constituted a subset of AT2 cells with a molecular signature indistinguishable from sorted Axin2^{CreERT2-Tdt}AT2 cells (FIGS. 20E-20L). Sorted Tfcp211-lineage AT2 cells efficiently form complex organoids indistinguishable from those generated by sorted Axin2-positive AEPs. In combination with recent reports showing a role for Tfcp211 in AT2 cells during injury repair, our data suggested that Tfcp211^{CreERT2} constituted an AEP-enriched inducible Cre line suitable for epithelial specific knockout of Nkx2-1.

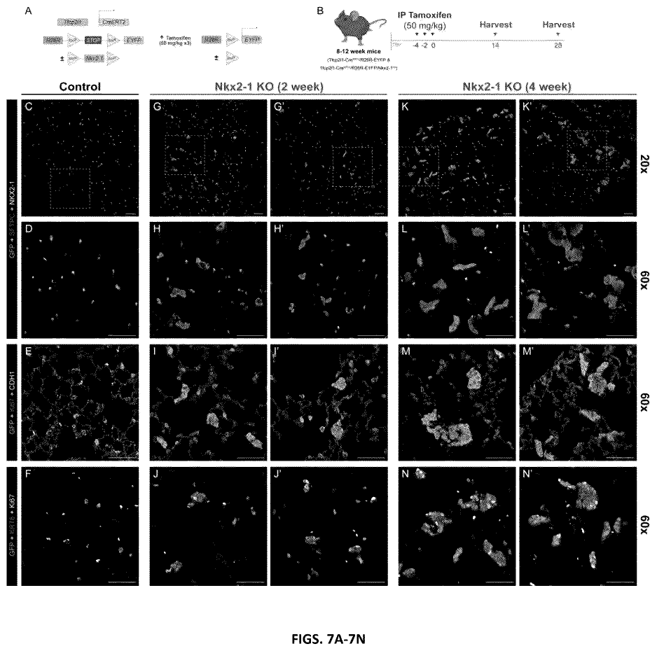

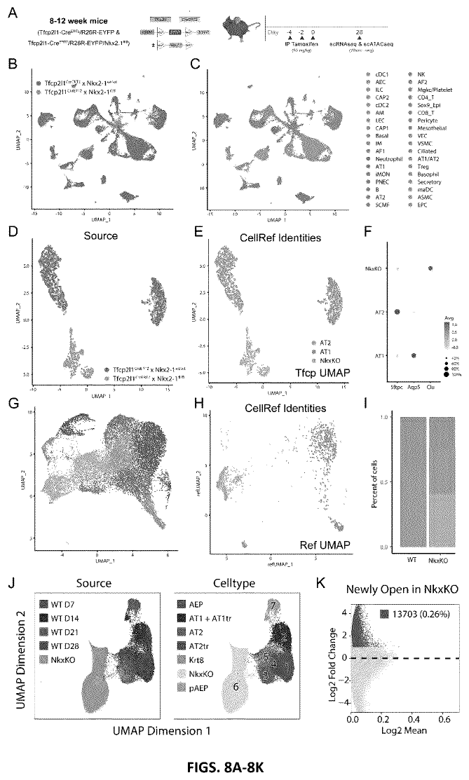

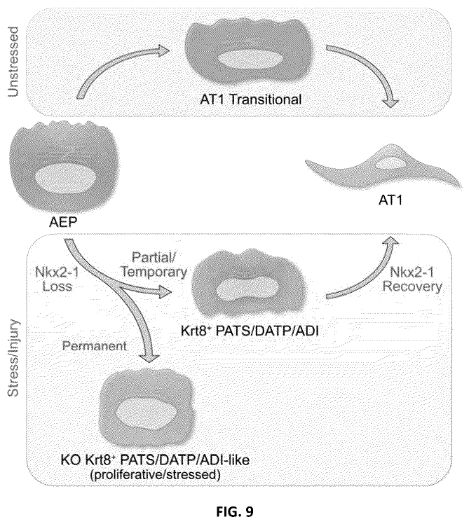

We therefore generated Tfcp211^{CreERT2}×R26R^{EYFP}×Nkx2-1^{flox/flox} animals, enabling Nkx2-1 knockout in AEPs during adult alveolar homeostasis (FIG. 7A-N). At 2 weeks following Nkx2-1 knockout in AEPs, we detected multi-cell clones of lineage labeled EYFP⁺, Nkx2-1^{KO} epithelial cells throughout the lung (FIG. 7F-I). Nkx2-1^{KO} Tfcp211-lineage cells in vivo lost expression of AT2 markers including Sftpc, underwent change in cell shape and morphology, and acquired an E-cadherin high, Krt8-expressing, proliferative state (FIG. 7J-M), consistent with changes seen in Nkx2-1^{KO} organoids. By 4 weeks of age, these Krt8⁺ clones have grown substantially, with persistent shape change and high-level proliferation (FIG. 7N-U). Disruption of alveolar morphology was present in some areas near larger clones (FIG. 7M). Next, we compared the molecular state of Nkx2-1^{KO} cells generated in vitro using AAV6.2FF-Cre in Axin2^{CreERT2-tdT}×R26R^{EYFP}×Nkx2-1^{flox/flox} and in vivo in Tfcp211^{CreERT2}×R26R^{EYFP}×Nkx2-1^{flox/flox}. We performed whole lung scRNAseq in Tfcp211^{CreERT2}×R26R^{EYFP}×Nkx2-1^{flox/flox} animals (FIGS. 8A-8C) and identified a Nkx2-1^{KO} specific distinct epithelial cell population which was lineage labeled with EYFP and expressed multiple markers of Krt8⁺-like cells (FIG. 8D-8F). We then evaluated the molecular signature of this population compared to those found in Nkx2-1^{KO} organoids and confirmed that this population clustered with Nkx2-1^{KO} cells from AEP-O and share extensive molecular similarity (FIG. 8G-H). Taken together, these findings confirm that, as in AEP-O, Nkx2-1 loss in AEPs in vivo drives acquisition of the Krt8⁺/PATS/DATP/ADI molecular state, with spontaneous proliferative growth and disruption of alveolar architecture (FIG. 9). In some embodiments, the AEPs are from Tfcp211^{CreERT2}×R26R^{EYFP}×Nkx2-1^{flox/flox} animals.

Combined scRNAseq and scATACseq of AEP-derived alveolar organoids allowed definition of regulatory networks along multiple differentiation trajectories of lung epithelial progenitor cells toward differentiated alveolar epithelium. Transcriptomic and epigenomic data demonstrated two distinct AEP to AT1 differentiation trajectories, one through a known stressed transitional cell state and another through a state defined by signaling integration and transcriptional regulators of AT1 cell gene programs. A major difference in the regulation of these two cell states was activity of Nkx2-1. Nkx2-1 expression in AEPs is important for maintaining the AEP state, and loss of Nkx2-1 activity is sufficient for AEPs to enter the Krt8⁺ stressed transitional state. Nkxk2-1 therefore plays an important, previously unrecognized role in the maintenance of progenitor function in the adult lung. These findings were enabled by the characteristics of the AEP-O system, allowing close interrogation of progenitor dynamics in a model with similar cellular and morphological complexity to alveolar regeneration in vivo.

### Under-Recognized Key Roles of Developmental Factors in Diverse Homeostatic and Regenerative Biology.

Nkx2-1 loss is sufficient to cause transition of AEPs to a proliferative stressed transitional state, a finding which emphasizes the need for active maintenance of adult AT2 alveolar progenitor capacity. Prior reports have demonstrated a requirement for Nkx2-1 expression in maintenance of lung epithelial fate in adult differentiated cells. Loss of one or more alleles of Nkx2-1 is a common mutation found in lung adenocarcinoma. At a deeper level, however, the finding that a single transcription factor in a facultative progenitor lineage is sufficient to drive not only a fate transition, but a conversion of a poised quiescent lineage to unconstrained proliferative growth in adult lung is quite provocative. It is increasingly understood that transcription factors exert influence on gene expression through changes in chromatin state at regulatory elements, especially so called “pioneer factors”. Nkx family factors are known to function as pioneer transcription factors in diverse contexts, and pioneer factors catalyze changes in chromatin structure that maintain epigenetic stability.

### Evaluation of Epigenomic State Enriches Single Cell RNA Evaluation of Multiple Similar Cell States.

The identification of Nkx2-1 as a regulator of AEP progenitor state relied on the ability to distinguish similar cell states in a dynamic system. Our data from AEP-O provided two useful adjuncts to traditional single cell analysis: known input/initial cell state in a clonal culture(obtained by sorting pure AEPs), and scATACseq to add epigenomic state data for comparison to matched RNA transcriptomes. These additions provided multiple benefits. First, the known initial cell state and clonal nature of AEP-O provided a clear ability to identify the starting point of differentiation. Identification of initial root cell in trajectory, pseudotime, and RNA velocity analyses can be challenging and the shortfalls can be mitigated by controlling input. Knowing the input cell state of AEP-O added confidence to algorithmic predictions when the unbiased methods correctly identified AEPs or proliferating AEPs as the initial state; multiple lines of evidence strengthened this conclusion and allow full analysis. Together, these factors enabled downstream identification of AT1 differentiation trajectories through both the AT1tr and Krt8⁺ states. Addition of chromatin state also provided a more refined signature for AEPs, allowing better identification of AEPs within the AT2 population, which has been challenging on the basis of scRNA transcriptome analysis alone. Together, scRNA and scATAC provided the resolution to derive TRN inference, leading to Nkx2-1.

### Transitional Cells at the Interface of Cell Stress, Regeneration, and Disease.

The marked differences between AT1tr and Krt8⁺ transitional cells further the complexity of choosing cellular targets for lung regeneration. Krt8⁺ stressed transitional cells have been described in diverse models, are found in mouse and human, are increased in several disease states, and are readily identifiable based on high level expression of enriched markers. However, our data shows that permanent acquisition of a stressed transitional state, such as seen in Nkx2-1^{KO} organoids and Nkx2-1^{KO} AEPs in vivo, drives aberrant proliferation, expression of lung cancer programs, and loss of lung identity. Progressive acquisition of this cell state is deleterious; even if most cells pass through or ‘recover’ from this stressed transitional state, accumulation of ‘stuck’ transitional cells represents a risk factor for development of lung disease.

### AEP-Os are a High-Fidelity Model of Alveolar Epithelial Regeneration in a Dish.

Development of new therapeutics to promote functional lung regeneration will benefit from high fidelity in vitro models of the regenerative process. Restoration of a gas exchange surface through repair or replacement of injured alveoli is the central process needed to promote therapeutic regeneration. Here, we show that AEP-derived lung organoids recapitulate the key aspects of the epithelial portion of the alveolar regenerative process, modeling progenitor cell expansion, alveolar epithelial differentiation, and formation of alveolar-like cavities with properly polarized and functional epithelium. Nkx2-1 deletion caused concordant changes both in organoids and in vivo, providing proof of principle that AEP-O model key aspects of adult alveolar biology and alveolar regeneration. The cultures described are generated from a clearly defined epithelial cell input, are clonal, and show high level reproducibility after optimization of matrix, mesenchymal components, and media conditions. Single cell analysis demonstrated that AEP-O recapitulate key aspects of in vivo epithelial differentiation and epithelial/mesenchymal interactions. TRN inference from these data implied a key role for Nkx2-1 in detected AT1 differentiation states, which was validated using both in vitro and in vivo lineage tracing and genetic targeting. These data suggest AEP-O will allow screening, molecular testing, and genetic manipulation via AAV6.2FF-Cre in a system which provides a balance between fidelity and reproducibility. While we characterized AEP-O only from mice in this study, in some embodiments the cells (AEP and/or mesenchyme cells) are human, resulting in human AEP-Os.

Controversy exists regarding the similarity of alveolar regeneration and alveolar development; some have argued that regeneration is fundamentally different due to the altered milieu of the injured alveolus, or even suggested that pathological remodeling rather than functional regeneration is the end state of a significant portion of lung injury. Our findings show that extensive regenerative potential is encoded in the AEP epigenomic state, driving a distinct process to rebuild alveoli. AEPs contain the required information to undergo progenitor self-renewal, multilineage differentiation, and complex morphogenesis in the presence of a minimal signaling niche. This challenge bears striking resemblance to the injured lung, where epithelial, mesenchymal, endothelial, immune lineages and the underlying matrix environment are all altered by pathogens. AEP-driven cavity formation occurs in the absence of mechanical contribution from myofibroblasts, quite different from during alveologenesis when the mechanical activity of myofibroblasts is required for formation of alveoli. Our data therefore emphasize the difference between alveolar development and alveolar regeneration and shows that AEP-O provide unique benefits to study regenerative biology specifically.

### Examples

### Materials and Methods

Ethical Compliance and Animals: All animal studies were conducted under the guidance and supervision of the Cincinnati Children's Hospital Medical Center (CCHMC) Institutional Animal Care and Use Committee (IACUC) in accordance with CCHMC regulatory and biosafety protocols. Mouse lines used included: C57BL/6J mice (Jackson Strain #000664), Pdgfrα^{EGFP} (B6.129S4-Pdgfra^{tm11(EGFP)Sor}/J; Jackson Strain #007669) Axin2^{creERT2-TdT} (a gift from Edward Morrisey), Tfcp211^{CreERT2} (B6;129S-Tfcp211^{tm1.1(cre/ERT2)oci}/J; Jackson Strain #028732), Nkx2-1^{ﬂ/ﬂ} (a gift from Shioko Kimura), and Rosa^{EYFP} (B6.129X1-Gt(ROSA)26Sor^{tm1(EYFP)Cos}/J; Jackson Strain #006148). All experiments for both organoids and in vivo lineage tracing included both male and female mice.

For Cre recombinase induction in mouse models, 8-12-week-old mice were treated intraperitoneally (IP) with Tamoxifen (Sigma, T5648; dissolved in ethanol and resuspended in corn oil) at a dose of 50 mg/kg, one or three times (every other day), at the experimental timepoints indicated previously.

Mouse Lung Harvest: Mice were anesthetized via intraperitoneal Ketamine+ Xylazine followed by euthanasia via cervical dislocation and thoracotomy. The chest cavity was opened to expose the heart and lungs. The right ventricle was perfused with 5-10 mL of cold PBS (Gibco, 10010-023) to clear blood from the lungs. For tissue dissociation for organoids, lungs were removed and placed in cold PBS on ice. For tissue fixation for histology and immunofluorescence, the trachea was cannulated and lungs were inflated to a pressure of 30 cm H₂O using 4% paraformaldehyde (PFA). Inflated lungs were immersed in a conical of 4% PFA, then left on a rocker at 4 °C overnight.

Processing Fixed Lung Tissue for Histology & Immunofluorescence: The day following inflation, fixed lung tissue was trimmed and placed in cassettes. The cassettes were washed (15 minutes each) 3× in DEPC-treated PBS, 1× in DEPC-treated 30% ethanol, lx in DEPC-treated 50% ethanol, and 3× in DEPC-treated 70% ethanol. Following a standardized overnight automated processing protocol (Thermo Scientific, Excelsior ES), the samples were embedded in paraffin. Samples were sectioned at a thickness of 5 μm. Paraffin sections were incubated at 65 °C for two hours, deparaffinized in xylene (3× for 10 minutes), rehydrated through an ethanol gradient, and standard H&E staining was performed. Slides were mounted with Permount Mounting Medium (Electron Microscopy Sciences, 17986-05) and cover slipped with #1.5 Gold Seal 3419 Cover Glass (Electron Microscopy Sciences, 63790-01). Immunofluorescence was on paraffin sections was performed as previously described using antibodies listed in Supplemental Table 1b and the following reagents ImmPRESS® HRP Horse Anti-Rabbit IgG Polymer Detection Kit (Vector Labs, MP-7401-50), ImmPRESS® HRP Horse Anti-Goat IgG Polymer Detection Kit (Vector Labs, MP-7405-50), and ImmPRESS® HRP Goat Anti-Rat IgG, Mouse adsorbed Polymer Detection Kit (Vector Labs, MP-7444-15). Following application of TSA fluorophores (listed in Supplemental Table 1b; 1:100), sections were stained with DAPI (Invitrogen, D1306; 1:1000) and mounted using Prolong Gold antifade mounting medium (Invitrogen, P36930).

Mouse Lung Digestion and Single Cell Suspension: Clonal mouse alveolar epithelial progenitor (AEP)-based alveolar organoids were generated as previously described with modifications (see Zacharias, W. J. et al. Regeneration of the lung alveolus by an evolutionarily conserved epithelial progenitor. Nature 555, 251-255, doi:10.1038/nature25786 PMID-29489752 (2018), herein incorporated by reference in its entirety). Briefly, following harvest, lungs were removed from ice cold PBS and non-pulmonary tissue and gross airways were removed via manual dissection, and lung tissue was finely chopped and transferred to a GentleMACS C tube (Miltenyi Biotec, 130-093-237) (tissue from one mouse per C tube) containing 5 mL of digestion buffer [composed of 9 mL of phosphate-buffered saline (PBS; Gibco, 10010-023) combined with 1 mL of Dispase (stock: 50 U/mL; final concentration: 5 U/mL, Coming, 354235), 50 μL of DNase (stock: 5 mg/mL; final concentration: 0.025 mg/mL or 50 U/ml, GoldBio, D-301), and 100 μL of Collagenase Type I (stock: 48,000 U/mL; final concentration of 480 U/mL, Gibco, 17100-017)]. C tubes were placed on a gentleMACS Octo Dissociator with Heaters (Miltenyi Biotec, 130-096-427) and the following protocols were run: “m_lung_01_02” (36 seconds) twice, “37C_m_LIDK_1” (36 minutes 12 seconds) once, and “m_lung_01_02” (36 seconds) once. Samples were passed through a 70 μm filter (Greiner Bio-One, 542070) and centrifuged at 500 g for 5 minutes at 4 °C. Following removal of the supernatant, 5 mL of RBC Lysis Buffer (Invitrogen, 00-4333-57) was added and incubated for 5 minutes. All centrifugation steps with this single cell suspension were performed at 500×g for 5 minutes at 4 °C for the following procedures.

Fibroblast Stock Preparation (with PDGFRα^{e}GFP fibroblasts)/Media: For generation of fibroblast stocks, 4-week C57BL/6J mice and 4-week PDGFRα^{eGFP} mice lungs were harvested, digested, and processed as described above. Following centrifugation, cells were washed 3× with MACS Buffer (autoMACS Rinsing Solution [Miltenyi Biotec, 130-091-222] with MACS BSA Stock Solution [Miltenyi Biotec, 130-091-376]). After removing supernatant from final wash, the cell pellet was resuspended in 10 mL fibroblast medium (DMEM/F-12 [Gibco, 11320-033], Antibiotic-Antimycotic [Gibco, 15240-062, final concentration 1×], and Heat Inactivated Fetal Bovine Serum [Corning, 35-011-CV, final concentration 10%]) and plated on a 10 cm tissue culture plate (approximately 1 mouse per plate). Non-adherent cells were removed via media change 2-12 hours post-plating.

Cells were passaged at 80% confluency to P3. For passaging, media was removed from each plate and cells were washed with 5 mL of DPBS (Gibco, 14190-094). 3 mL of 0.25% Trypsin-EDTA (Gibco, 25200-056) was added and plates were incubated at 37 °C for 7 minutes. 5 mL of fibroblast medium was added to each plate, pipetted to dissociate cells, and transferred a 15 mL conical tube. Cells were centrifuged at 500 g for 5 minutes at 4 °C, supernatant was removed, and cell pellet was resuspended in 2 mL fibroblast medium/per plate (split 1:2 or 1:3) and transferred to plates containing 6 mL fibroblast medium.

Once confluent at P3, cells were washed, trypsinized, centrifuged as above, and resuspended in 1 mL of freezing medium (90% FBS, 10% DMSO) (one plate per cryovial) and transferred to Mr. Frosty Cryogenic Freezing Container (Nalgene, 5100-0001) filled with isopropyl alcohol, which were placed in a −80 °C freezer overnight, before samples were moved to long-term liquid nitrogen storage.

For use of frozen fibroblast stocks in organoids, 48 hours prior to use in organoids, cells were rapidly thawed and resuspended in 10 mL fibroblast medium in a 15 mL conical. Cells were centrifuged at 500 g for 5 minutes at 4 °C, supernatant was removed, and cell pellet was resuspended in 2 mL fibroblast medium and transferred to a 10 cm tissue culture plate containing 6 mL of fibroblast medium. Fibroblasts used for organoids were washed, trypsinized, and resuspended (as described for passaging) before counting.

Processing for Organoids/FACS/Cell Sorting: Single cell suspensions were obtained as above, and cells were resuspended in 5 mL MACS Buffer (autoMACS Rinsing Solution [Miltenyi Biotec, 130-091-222] with MACS BSA Stock Solution [Miltenyi Biotec, 130-091-376]) and passed through a 40 μm filter (Greiner Bio-One, 542040). Cells were centrifuged, the supernatant was removed, and the cell pellet was resuspended in Fc Receptor Binding Inhibitor Polyclonal Antibody (Invitrogen, 14-9161-73) diluted 1:100 in MACS buffer and incubated for 10 minutes at room temperature. Following centrifugation, cells were resuspended in a mixture of the following antibodies diluted 1:100 in MACS buffer and incubated for 10 minutes protected from light: CD31 (PECAM-1; Monoclonal Antibody [390], eFluor 450) (Invitrogen, 48-0311-82), CD45 (Monoclonal Antibody [30-F11], eFluor 450) (Invitrogen, 48-0451-82), CD326 (EpCAM; Monoclonal Antibody [G8.8], APC) (Invitrogen, 17-5791-82). Cells were washed 1× with 1-5 mL of MACS buffer, and resuspended in Fixable Viability Dye eFluor 780 (Invitrogen, 65-0865-14) diluted 1:1000 in MACS buffer and incubated for 15 minutes protected from light. Cells were centrifuged and washed in 1-5 mL MACS buffer 3×. After the final wash/centrifugation, the cell pellet was resuspended in MACS buffer (volume adjusted for cell count) and passed through a 35 μm filter lid (Coming, 352235) into a FACS tube for sorting.

Using single-stain controls from experimental animals and wild-type littermates (TdTomato-) for compensation and adjusting gating to remove debris/doublets, the live/CD31⁻/CD45⁻/CD326⁺ (EpCAM⁺)/TdTomato⁺ (AEP) population was sorted into a tube containing ‘spiked’ SAGM organoid medium (see ‘Organoid Medium’ section below) at 4 °C, using a BD FACSAria Fusion cell sorter with a 100 μm nozzle. Approximately 10′ AEPs have been sorted from one mouse using this protocol.

Organoid Medium: To generate ‘spiked’ SAGM medium for mouse lung alveolar organoids, SABM Small Airway Epithelial Cell Growth Basal Medium (Lonza, CC-3119) was combined with the following additives: SAGM Small Airway Epithelial Cell Growth Medium SingleQuots Supplements and Growth Factors (using only the BPE [2 mL], Insulin [0.5 mL], Retinoic Acid [0.5 mL], Transferrin [0.5 mL], and hEGF [0.5 mL] aliquots) (Lonza, CC-4124), Heat Inactivated Fetal Bovine Serum (Corning, 35-011-CV, final concentration 5%), Antibiotic-Antimycotic (Gibco, 15240-062, final concentration 1×), Cholera Toxin from Vibrio cholerae (Sigma, C8052, final concentration 25 ng/mL).

Standard Organoid Plating/Maintenance: AEPs (live/CD31⁻/CD45⁻/CD326⁺[EpCAM⁺]/TdT⁺ cells) sorted from Axin2^{CreERT2-tDT} mice were counted using a hemocytometer and resuspended in ‘spiked’ SAGM at a concentration 500 cells/μL. Fibroblasts were prepared (as described above), counted, and resuspended in ‘spiked’ SAGM at a concentration of 5000 cells/μL. For the remaining steps, it was extremely important that all reagents are kept cold/on ice and that bubbles were not introduced to mixtures when pipetting. It is recommended to prepare the plate (Falcon 24 well companion plates [Corning, 353504]), insert transwells (Falcon Transwell Insert/Permeable Support with 0.4 μm membrane [Corning, 353095]), and place them on ice before use.

For each well of organoids to be plated, 10 μL AEPs (5000 total cells), 10 μL fibroblasts (50000 total cells), and 25 μL ‘spiked’ SAGM were combined (create one master mix of cells and medium for all wells before adding Matrigel) and placed on ice. Corning Matrigel GFR Membrane Matrix (Corning, 356231) was added to the cell mixture (45 μL per well, 1:1 ratio of SAGM to Matrigel) and carefully mixed, then placed back on ice.

For plating, 90 μL of the combined cell/Matrigel mixture was pipetted carefully directly into the center of the transwell (placed in the companion plate) without introducing bubbles. Organoid plates were incubated at 37 °C for 15 minutes, then 500 μL of ‘spiked’ SAGM supplemented with ROCK Inhibitor/Y-27632 Dihydrochloride (Sigma, Y0503, final concentration 0.01 mM) was added beneath the transwell insert. After 48 hours (and for subsequent media changes), media was replaced every 2 days with ‘spiked’ SAGM without ROCK inhibitor and plates were maintained at 5% CO2 and 37 °C.

AAV6.2FF-Cre Organoids Plating/Maintenance: AAV6.2FF-Cre (titer of 2.779×10¹⁰ viral genomes [vg]/μL) was generated and characterized in vivo as previously described. Working dilutions (2.779×10⁹ vg/μL, 2.779×10⁸ vg/μL, and 2.779×10⁷ vg/μL) were generated via serial dilution of viral stocks in ‘spiked’ SAGM and frozen −80 °C in single use aliquots.

AEPs (live/CD31⁻/CD45⁻/CD326⁺[EpCAM⁺]/TdT⁺ cells) sorted from Axin2^{CreERT2-tDT}; R26R^{EYFP} mice were counted using a hemocytometer and resuspended in ‘spiked’ SAGM at a concentration 1000 cells/μL. The total cells needed for the desired number of wells were transferred to a new 1.5 mL tube (i.e., 10 wells→50000 cells→50 μL cells [1000 cells/μL]). Total cell number per tube, desired MOI (i.e., 1000, 10000, 20000), and known viral titers were used to calculate the volume of virus needed from viral stocks. For each MOI, the calculated volume of virus was added to each cell mixture, mixed, and incubated on ice for 60 minutes. Following viral incubation, ‘spiked’ SAGM and fibroblasts (5000 cells/μL) were added to create a mixture with the same proportions of cells as described above for standard plating of organoids (i.e., for each well—5000 AEPs+50000 fibroblasts in 45 μL ‘spiked’ SAGM). The cell mixture was mixed with Matrigel (45 μL/well) and plated/maintained as described above for standard organoids.

Organoid Plating with Fibroblasts on Basolateral Side of Transwell: One day prior to organoid plating, fibroblasts were prepared (as described above), counted, and resuspended at a concentration of 50000 cells in 100 μL in fibroblast medium. Transwells were placed in the wells of the companion plate, then the plate was flipped so the transwells rested on the inside of the lid. The companion plate was removed exposing the basolateral side of the transwells/filters. 100 μL of the resuspended fibroblast mixture was added to the basolateral side of each transwell filter. The plate base was placed back on top of the transwells, and was incubated (basolateral side up) at 37 °C and 5% CO2 for 4 hours. After incubation, the 100 μL of medium was removed via gentle pipetting (without disturbing the filter) and the plate was flipped to the standard orientation. The transwells were washed with 500 μL of DPBS (beneath the transwell insert) then moved to a fresh well/plate with 500 μL fibroblast medium beneath the transwell insert. After standard isolation of epithelial cells (AEPs) for organoid plating, the transwells were again washed with 500 μL of DPBS (beneath the transwell insert), then the epithelial cell/Matrigel mixture (5000 AEPs in 45 μL ‘spiked’ SAGM+45 μL Matrigel) was added to the apical side of the transwell filter. Organoids were maintained as described above for standard organoids.

Fixation/Processing for Sections/Histology/H&E: Organoids were washed with 500 μL PBS, above and below transwells. After removing PBS, 500 μL of 4% PFA was added above and below the transwell for fixation overnight at 4 °C. Transwells were washed 5×(above and below) with PBS. Using a small knife or scalpel, the transwell filter and Matrigel plug/organoids were cut out of the transwell and placed on parafilm. Using forceps, the transwell filter was carefully removed from the Matrigel plug/organoids [note: older organoid cultures are more likely to adhere to the filter]. Using a transfer pipet, HistoGel (Epredia, HG4000012) (pre-heated to a liquid consistency) was added on top of the Matrigel plug/organoids until covered on all sides. Once solidified (~15-30 minutes), the sample was transferred to a tissue processing cassette (Fisher, 15-182-702A). Once in cassettes, samples were processed as described for whole-lung processing above for paraffin embedding and sectioning, H&E, and immunofluorescence of paraffin sections.

Isolation of Organoids for Whole-Mount Immunofluorescence: Previously established whole-mount organoid staining protocols from Dekkers, et al. were adapted for mouse lung alveolar organoids using the following modifications. All steps used cut or wide-bore pipette tips. All steps after first wash and prior to fixation were performed on ice/with chilled reagents and used cut/wide bore pipette tips coated in 1% BSA in PBS.

Briefly, transwells were washed (above and below) with 500 μL of room temperature PBS. 500 μL of Cell Recovery Solution (Corning, 354253) was added to each transwell, and a cut pipette tip was used to mechanically disrupt the Matrigel—the mixture was pipetted up and down and transferred to a new 24 well plate. Each transwell was washed with an additional 250 μL of cell recovery solution and added to the new plate. The plate was incubated on ice (gel ice packs were optimal) on an orbital/horizontal shaker for 60 minutes. The organoid mixture was transferred to a 15 mL conical pre-coated in 1% PBS-BSA. Each well was washed with 500 μL of 1% PBS-BSA and added to the 15 mL conical. Wells from the same experimental condition (up to 4 wells) were combined in one conical. Conicals were filled to 10 mL with ice cold PBS and centrifuged at 70 g for 5 minutes at 4 °C. The supernatant was removed very carefully [note: if the organoid pellet is not compact/tight, the entire pellet may be lost with suction due to loose matrix]. If Matrigel was still visible, the organoid pellet was gently resuspended in 1 mL of ice cold 1% PBS-BSA and centrifuged again at 70 g for 5 min at 4 °C. After careful removal of the supernatant, the organoid pellet was resuspended in 1 mL of 4% PFA and incubated at 4 °C for 45 minutes (resuspending once halfway through incubation). For permeabilization, conicals were filled to 10 mL with 0.1% PBS-Tween and incubated overnight at 4 °C (alternate permeabilization option: for Click-iT protocols or shorter permeabilization, remove PFA and incubate in 0.25% Triton X-100 for 20 minutes at room temperature).

Whole-Mount Blocking and Immunofluorescence: After isolation, fixation, and permeabilization, organoids were centrifuged at 70 g for 5 min at 4 °C, resuspended in 500 μL of 5% Normal Donkey Serum (Jackson ImmunoResearch, 017-000-121) in 0.1% PBS-Triton X-100, and transferred to a 24-well plate for blocking. Organoids were incubated at room temperature on an orbital shaker for 1-2 hours.

After blocking, the supernatant was removed from each well without disturbing organoids [note: supernatant was removed more easily when plate was placed at a 450 angle for 5-10 minutes, to allow organoids to settle to bottom edge of well]. Primary antibodies (see supplemental table 1) were diluted to a final concentration of 1:100 in 5% Normal Donkey Serum in 0.1% PBS-Triton X-100 (approximately 200-250 μL total) and incubated overnight at 4 °C on an orbital shaker. For this protocol, a ‘quick wash’ was defined as adding 1 mL of organoid wash buffer (0.2% BSA, 0.1% Triton X-100 in PBS) and immediately allowing organoids to settle/removing the wash, and a ‘long wash’ was defined as adding 1 mL of organoid wash buffer placing the plate on an orbital shaker for 1-2 hours before allowing organoids to settle/removing the wash. After primary antibody staining, one ‘quick wash’ and three ‘long washes’ were performed. Then, secondary antibodies (see supplemental table 1) were diluted to a final concentration of 1:200 in 5% Normal Donkey Serum in 0.1% PBS-Triton X-100 (approximately 200-250 μL total) and incubated overnight at 4 °C on an orbital shaker. For this step and all subsequent steps, samples were covered/protected from light to prevent photobleaching. After secondary antibody staining, one ‘quick wash’ was performed, then organoids were incubated in DAPI (Invitrogen, D1306, final concentration of 1:1000) in 5% Normal Donkey Serum in 0.1% PBS-Triton X-100 (approximately 200-250 μL total) for 15 minutes. After removing supernatant, one ‘quick wash’ and three ‘long washes’ were performed.

Whole-Mount Clearing and Mounting: Following the final wash after immunostaining, as much wash buffer as possible was removed from each well and organoids were transferred to a 1.5 mL tube. Organoids were centrifuged at 70 g for 5 min at 4 °C and as much supernatant as possible was removed without disturbing the organoids. Using a cut or wide-bore pipette tip, organoids were gently resuspended in room temperature fructose-glycerol clearing solution (60% vol/vol glycerol+2.5 M fructose). Depending on organoid volume, ~50-200 μL of clearing solution was used. Organoids were left to clear for at least 1 day (and as long as several months) at 4 °C before mounting.

Prior to preparing slides, cleared organoids were allowed to equilibrate to room temperature. Organoids were mounted as described previously—briefly, two pieces of double-sided tape were applied to a microscope slide approximately 25-30 mm apart, perpendicular to the length of the slide (for larger organoids, additional layers of tape can be used). Using a PAP pen (Abcam, ab2601), a square was drawn between the two pieces of tape. Using a cut P200 pipette tip, approximately 20 μL of organoids in clearing solution was placed in the middle of the drawn square, avoiding bubbles. A #1.5 Gold Seal 3419 Cover Glass (Electron Microscopy Sciences, 63790-01), was applied over the organoids, bridging the two pieces of tape. Slides were imaged immediately or stored at 4 °C.

Whole-Mount Click-iT EdU Staining: For whole-mount Click-iT EdU staining, the standardized commercial protocol for Click-iT EdU Cell Proliferation Kit for Imaging, Alexa Fluor 488 dye (Invitrogen, C10337) was combined with our optimized whole-mount immunofluorescence protocol. Kit reagents were prepared as directed in commercial protocols. Briefly, 48 hours prior to harvest/fixation, organoid medium was replaced with ‘spiked’ SAGM supplemented with EdU (final concentration of 10 μM) from the commercial kit. After EdU incubation, all steps described in ‘Isolation of Organoids for Whole-Mount Immunofluorescence’ were performed. Next, steps for EdU detection from the commercial kit's standardized protocol with kit reagents were followed (i.e., 30-minute incubation of “Click-iT Reaction Cocktail” at room temperature, followed by 1 mL wash with 3% PBS-BSA). Following EdU detection, whole-mount immunofluorescence was performed as described in the ‘Whole-Mount Blocking and Immunofluorescence’ and ‘Whole-Mount Clearing and Mounting’ sections above.

Whole-Mount Click-iT TUNEL Staining: For whole-mount Click-iT TUNEL staining, the standardized commercial protocol for Click-iT Plus TUNEL Assay for In Situ Apoptosis Detection, Alexa Fluor 488 dye (Invitrogen, C10617) was combined with our optimized whole-mount immunofluorescence protocol. Kit reagents were prepared as directed in commercial protocols. All steps described in ‘Isolation of Organoids for Whole-Mount Immunofluorescence’ were performed. Organoids were washed twice with DI H2O. Next, steps for ‘TdT Reaction’ and ‘Click-iT Plus Reaction’ from the commercial kit's standardized protocol with kit reagents were followed (i.e., 60-minute incubation of “TdT Reaction Mixture” at 37 °C, followed by 2 washes with 3% PBS-BSA, and a 30-minute incubation of “Click-iT Plus TUNEL reaction cocktail” at 37 °C). Organoids were washed twice with 3% PBS-BSA, then whole-mount immunofluorescence was performed as described in the ‘Whole-Mount Blocking and Immunofluorescence‘ and ‘Whole-Mount Clearing and Mounting’ sections above.

Hoechst and Live Imaging Preparation: For live imaging of organoids grown with PDGFRαeGFP fibroblasts, Hoechst 33342 (Invitrogen, H3570) was diluted 1:10000 in ‘spiked’ SAGM and 500 μL was added above and below the transwell and incubated at 37 °C for 30-45 minutes. Using a small knife or scalpel, the transwell filters and Matrigel plug/organoids were cut out of the transwells and placed into a coverslip bottom dish (MatTek, P35G-1.5-20-C). For some samples, the entire Matrigel plug/filter was imaged, and for others the Matrigel plug and filter were separated and imaged independently. Samples were covered in ‘spiked’ SAGM and coverslipped (MatTek, PCS-1.5-18) prior to imaging on an inverted confocal microscope.

Imaging: Brightfield H&E images were acquired on a Nikon Eclipse NiE Upright Widefield Microscope (Nikon DS-Fi3 Camera—with a Plan Apo VC 20×DIC N2 objective). Fluorescent images were acquired on Nikon Al inverted LUNV and Nikon AiR inverted LUNV confocal microscopes using the following objectives: Plan Apo λ 10×, Plan Apo λ 20×, Apo LWD 20×WI πS (water immersion), Apo LWD 40×WI λS DIC N2 (water immersion), and SR HP Plan Apo λ S 100×C Sil (silicone immersion). Second harmonics images were obtained using a Nikon FN1 Upright Multiphoton microscope using the following objectives: Plan Apo VC 20×DIC N2 and Apo LWD 25×1.10W DIC N2. Images were processed in Nikon Elements with minimal, global adjustment of LUTs for acquired channels.

Organoid Plate Imaging/Cytation Imager: For whole well imaging, plates were loaded into a Cytation 5 Imager (BioTek, CYT5PV) configured with a CO2 gas controller (BioTek, 1210012). Plates were maintained at 5% CO2 and 37 °C during imaging using Cytation Gen5 Microplate Reader and Imager Software (BioTek, version 3.08.01). Protocols specific to Falcon 24 well companion plates (Corning, 353504) and Falcon Transwell Insert/Permeable Support with 0.4 μm membrane (Corning, 353095) were established and used to take brightfield and fluorescent (GFP) 4×tile scans at 10 z-steps (~50 μm per step). 4×tile scans were used to generate z-projections. Individual tile scans and z-projections were used for further quantification.

Organoid Quantification: Z-projections of stitched 4×images from each well were loaded into a custom FIJI-macro (run in FIJI/ImageJ v1.53) to count organoids per well, GFP⁺ organoids per well, and organoid area. This macro allowed for batch analysis of each experiment, reducing subjectivity of counts. Briefly, given specific input parameters, the macro contained commands to: set the scale based on the diameter of each transwell, subtract background, adjust image threshold, convert to mask, analyze particles/count objects meeting a specific threshold, and export data. Data was imported into GraphPad Prism 9.0 for analysis. T tests were used for comparison of 2 groups, and ANOVA with prespecified multiple comparisons was used to compare 3 or more groups.

Electron Microscopy: Fixation, sectioning, and acquisition of electron micrographs of alveolar cells was performed as previously described.

Organoid Dissociation and Preparation of Single Cell Suspension for scRNA-seg and scATAC-seq: Transwells were washed (above and below) with 1 mL of PBS. 60 μL of organoid digest buffer (Dispase [Corning, 354235, undiluted, 50 U/mL], DNase I [GoldBio, D-301, final concentration 5 U/mL], Collagenase Type I [Gibco, 17100017, final concentration 4800 U/mL)]) was added and Matrigel plugs were gently disrupted and pipetted using a cut or wide-bore pipette tip. Organoids were incubated in digest buffer for 30 minutes at 37 °C. Following incubation, the digested organoid mixture was pipetted several times and transferred to a low-binding 1.5 mL tube (3 wells of same experimental condition combined into each tube). 500 μL of cold PBS was added to each tube and incubated on ice for 5-10 minutes. Samples were centrifuged at 500 g for 5 minutes at 4 °C. Supernatant was removed carefully and sample was washed with 1 mL of cold DPBS (Gibco, 14190-094). Following centrifugation at 500 g for 5 minutes at 4 °C and removal of supernatant, samples were resuspended in 60 μL of 0.25% Trypsin-EDTA (Gibco, 25200-056) and incubated for 30 minutes at 37 °C. 1 mL of ice-cold PBS was added to each tube, samples were centrifuged at 500 g for 5 minutes at 4 °C, and the supernatant was carefully removed. Samples were washed 2× in 1 mL of cold 0.04% PBS-BSA and centrifuged at 500 g for 5 minutes at 4 °C. Following removal of the supernatant, the samples were resuspended in 100 μL of 0.04% PBS-BSA. Prior to filtering cells, 40 μm Flowmi Cell Strainers (Bel-Art, H13680-0040) were equilibrated by passing 100 μL of 0.04% PBS-BSA through the strainer using a P1000 pipette tip. The 100 μL cell suspension was then pipetted through the 40 μm Flowmi Cell Strainer. Cells were counted manually using a hemocytometer and resuspended at a concentration of 1000 cells/μL prior to processing for scRNA-seq.

Nuclei Isolation from Organoids for scATAC-seq: Using the same filtered cell suspension generated for scRNA-seq, the standard 10× Genomics protocol for ‘Nuclei Isolation for Single Cell ATAC Sequencing’ (CG000212 Revision B) was followed. Briefly, the single cell suspension was centrifuged at 500 g for 5 minutes at 4 °C. Following removal of the supernatant, 100 μL of ATAC lysis buffer (from standard 10× Genomics protocol, CG000212 Revision B) was added, gently mixed, and incubated on ice for 4-4.5 minutes. Immediately following incubation, 1 mL of chilled ATAC wash buffer (from standard 10× Genomics protocol) was added and gently mixed. Nuclei were centrifuged at 500 g for 5 minutes at 4 °C, supernatant was removed, and nuclei were resuspended in 100 μL of 1× nuclei buffer (diluted from 20×nuclei buffer [10× Genomics, 2000153/2000207]). Prior to filtering nuclei, 40 μm Flowmi Cell Strainers were equilibrated by passing 100 μL of nuclei buffer through the strainer using a P1000 pipette tip. The 100 μL of nuclei suspension was then pipetted through the 40 μm Flowmi Cell Strainer. Nuclei were counted manually using a hemocytometer and resuspended at a concentration of 5000 nuclei/μL prior to processing for scATAC-seq.

Sequencing/Library Preparation: From each single cell or single nuclear preparation described above, a maximum of 16,000 cells or nuclei were loaded into on channel of a 10× Genomics Chromium system by the Cincinnati Children's Hospital Medical Center Single Cell Sequencing Core. Libraries for RNA (v3) and ATACseq (v2) were generated following the manufacturer's protocol. Sequencing was performed by the Cincinnati Children's Hospital DNA Sequencing Core using Illumina reagents. Raw Sequencing data was aligned to the mouse reference genome mm10 with CellRanger 3.0.2 to generate expression count matrix files. To detect YFP expressing cells following Cre-mediated activation, a YFP contig was added to the mm10 genome following 10× Genomics “Build a Custom Reference” instructions(https://support.1Oxgenomics.com/single-cell-gene-expression/software/pipelines/latest/using/tutorial_mr) with modifications. Briefly, a custom EYFP.fasta file was generated using the EYFP segment (682-1389) of the pEYFP-N1 plasmid sequence available through Addgene. This sequence was integrated into the standard mm10 assembly available from Ensembl to create a reference compatible for alignment with the CellRanger pipeline described above.

scRNAseq Analysis and Visualization: For RNAseq analysis, output data from CellRanger was partitioned into spliced and unspliced reads using Velocyto. Velocyto output files were loaded into Seurat 4.0 using SeuratWrappers and SeuratDisk using the ReadVelocity command and spliced transcripts were used as the expression input to SCTransform. Cells with less than 2000 or more than 8000 features were filtered and cells were clustered using the standard Seurat workflow. Putative doublets were identified and removed using DoubletFinder, and libraries from individual time points and treatments were integrated using SelectlntegrationFeatures and IntegrateData commands in Seurat. Following integration, cells were re-clustered, UMAP project generated, and samples identified based on expression similarity to published data as described in the Results. Module scoring was performed using AddModuleScore function in Seurat for gene sets indicated in the figures. For lineage inference, these Seurat objects were directly used for Slingshot pseudotime inference, and were converted to a h5ad file using the SaveH5Seurat command. These h5ad file were used as input to scVelo and CellRank in Python 3.9.12 in Spyder following the standard pipeline (scVelo.readthedocs.io) to generate RNA velocity mapped to the Seurat UMAP and cell populations. For ligand-receptor analysis, we used CellChat (https://github.com/sqjin/CellChat) v1.1 using the SecretedSignaling subset of the Mouse CellChatDB, with default parameters. Visualizations were generated with these tools and ggplot2.

For ATACseq analysis, CellRanger output was loaded into ArchR and Arrow files were generated per package defaults. Clusters were generated based on ATACseq parameters and named based on evaluation of integrated gene expression from the paired Seurat RNA object. Peak calls for regions of open chromatin were generated from pseudobulk analysis of each cell state followed by peak calling in MACS2. Differential open chromatin peaks were identified based on FDR<0.01 and Log2FC>=1 between cell states. Visualizations were generated using standard ArchR commands.

Transcriptional Regulatory Networks and Visualization: For TRN inference, scRNAseq gene expression data and scATACseq chromatin accessibility data from each epithelial cell population was used as input for the Inferolater 3.0 (github.com/flatironinstitute/inferelator) in Python 3.9.22. Enriched transcriptional regulators were identified per cell type by performing Fisher's Exact Test to compare observed vs expected number of genes regulated in a cell type based on the TRN model, and visualizations were generated in Illustrator with details as noted in the Figure Legends.

SUPPLEMENTAL TABLE 1a
Organoid Whole-Mount Immunofluorescence Antibodies:

| Primary Antibody (1:100 - unless specified otherwise) | Secondary Antibody (1:200) |
|---|---|
| anti-Sftpc (Guinea Pig, Seven Hills Bioreagents, GP992) | Goat anti-Guinea Pig IgG Alexa Fluor 555 (Invitrogen, A21435) |
| | Goat anti-Guinea Pig IgG Alexa Fluor 647 (Invitrogen, A21450) |
| anti-RAGE (Rat, R&D Systems, MAB1179) | Donkey anti-Rat IgG Alexa Fluor 488 (Invitrogen, A21208) |
| | Donkey anti-Rat IgG Alexa Fluor 568 (Abcam, ab175708) |
| anti-TTF1/Nkx2-1 (Rabbit, Seven Hills Bioreagents, Rb1231) | Donkey anti-Rabbit IgG Alexa Fluor 647 (Invitrogen, A31573) |
| anti-Ki67 (Rabbit, Abcam, ab16667 [SP6]) | Donkey anti-Rabbit IgG Alexa Fluor 647 (Invitrogen, A31573) |
| anti-GFP (Chicken, Abcam, ab 13970) | Goat anti-Chicken IgG Alexa Fluor 488 (Invitrogen, A11039) |
| anti-CD45 (Rat, BioLegend, 103103) | Rabbit anti-Rat IgG Alexa Fluor 488 (Invitrogen, A21210) |
| anti-E-Cadherin (Rat, R&D Systems, MAB7481 - 1:250) | Donkey anti-Rat IgG Alexa Fluor 568 (Abcam, ab175708) |
| anti-Krt8 (Rat, DSHB, TROMA-I - 1:50) | Donkey anti-Rat IgG Alexa Fluor 568 (Abcam, ab175708) |

SUPPLEMENTAL TABLE 1b
Paraffin Section Immunofluorescence Antibodies:

| Primary Antibody | Secondary Detection |
|---|---|
| anti-GFP (Rabbit, Abcam, ab290 - 1:1000) | TSA Plus Fluorescein (Akoya Biosciences, NEL741001KT) |
| anti-RAGE (Rat, R&D Systems, MAB1179 - 1:100) | |
| anti-Sox2 (Rabbit, Seven Hills Bioreagents Rb1236 - 1:200) | |
| anti-Sox9 (Rabbit, Sigma Aldrich, AB5535 - 1:100) | |
| anti-Cdx2 (Rabbit, Cell Marque, EPR2764Y - 1:500) | |
| anti-Gata4 (Rabbit, Santa Cruz, sc-9053 - 1:200) | |
| anti-Pdx1 (Goat, Abcam, ab47383 - 1:5000) | |
| anti-Krt8 (Rat, DSHB, TROMA-I - 1:200) | TSA Plus Cyanine 3.5 (Akoya Biosciences, NEL763001KT |
| anti-Sftpc (Rabbit, Seven Hills Bioreagents, Rb09337 - 1:250) | |
| anti-Ki67 (Rabbit, Abcam, ab16667 [SP6] - 1:100) | TSA Plus Cyanine 3.5 (Akoya Biosciences, NEL763001KT) |
| anti-E-Cadherin (Rat, R&D Systems, MAB7481 - 1:1000) | TSA Plus Cyanine 5 (Akoya Biosciences, NEL745001KT) |
| anti-TTF1/Nkx2-1 (Rabbit, Seven Hills Bioreagents, Rb1231 - 1:100) | TSA Plus Fluorescein (Akoya Biosciences, NEL741001KT) |
| | TSA Plus Cyanine 5 (Akoya Biosciences, NEL745001KT) |

SUPPLEMENTAL TABLE 2
scRNA-Seq Output Summary:

| | | | | Day 28 | |
|---|---|---|---|---|---|
| | | | | AAV | Day 28 |
| Sample ID | Day 14 | Day 21 | Day 28 | Control | Nkx2-1 |
| Estimated Number of Cells | 9,908 | 9,519 | 5,546 | 9,184 | 11,057 |
| Mean Reads per Cell | 40,509 | 42,230 | 69,615 | 63,819 | 44,557 |
| Median Genes per Cell | 3,606 | 3,637 | 4,431 | 2,766 | 2,925 |
| Number of Reads | 401,364,390 | 401,993,978 | 386,086,102 | 586,113,691 | 492,668,484 |
| Valid Barcodes | 97.9% | 98.2% | 98.2% | 97.60% | 97.70% |
| Sequencing Saturation | 23.4% | 28.9% | 41.9% | 28.60% | 28.10% |
| Q30 Bases in Barcode | 95.8% | 95.8% | 95.8% | 96.70% | 96.70% |
| Q30 Bases in RNA Read | 93.3% | 93.6% | 93.7% | 94.20% | 93.90% |
| Q30 Bases in Sample Index | 93.0% | 94.5% | 94.6% | | |
| Q30 Bases in UMI | 95.4% | 95.4% | 95.4% | 96.20% | 96.20% |
| Reads Mapped to Genome | 90.9% | 91.9% | 93.0% | 82.80% | 85.60% |
| Reads Mapped Confidently to | 86.7% | 88.0% | 89.2% | 78.30% | 81.50% |
| Genome | | | | | |
| Reads Mapped Confidently to | 6.0% | 6.1% | 7.2% | 8.50% | 12.20% |
| Intergenic Regions | | | | | |
| Reads Mapped Confidently to | 12.7% | 11.1% | 10.4% | 11.80% | 10.40% |
| Intronic Regions | | | | | |
| Reads Mapped Confidently to | 67.9% | 70.7% | 71.6% | 58.00% | 58.90% |
| Exonic Regions | | | | | |
| Reads Mapped Confidently to | 64.5% | 67.7% | 68.8% | 55.20% | 56.40% |
| Transcriptome | | | | | |
| Reads Mapped Antisense to | 2.0% | 1.6% | 1.4% | 1.90% | 1.20% |
| Gene | | | | | |
| Fraction Reads in Cells | 76.7% | 78.2% | 83.7% | 48.90% | 84.40% |
| Total Genes Detected | 20,300 | 20,134 | 19,308 | 22,830 | 17,723 |
| Median UMI Counts per Cell | 13,384 | 14,538 | 22,287 | 9,140 | 10,571 |

SUPPLEMENTAL TABLE 3
scATAC-Seg Output Summary:

| Sample ID | Day 14 | Day 21 | Day 28 |
|---|---|---|---|
| Annotated Cells | 13,300 | 11,765 | 4,535 |
| bc_q30 Bases Percent | 89.4% | 90.0% | 78.4% |
| Cellranger-ATAC Version | 1.2.0 | 1.2.0 | 1.2.0 |
| Cells Detected | 13,300 | 11,765 | 4,535 |
| Percent Cut Fragments in Peaks | 47.0% | 34.7% | 60.3% |
| Percent Fragments nfr | 67.8% | 71.0% | 68.6% |
| Percent Fragments nfr or nuc | 94.4% | 96.1% | 93.2% |
| Percent Fragments nuc | 26.6% | 25.1% | 24.6% |
| Percent Fragments Overlapping Peaks | 49.1% | 36.7% | 62.8% |
| Percent Fragments Overlapping Targets | 63.4% | 55.6% | 74.6% |
| Percent Mapped Confidently | 79.3% | 76.9% | 69.3% |
| Percent Waste Chimeric | 0.6% | 0.4% | 0.5% |
| Percent Waste Duplicate | 13.6% | 7.7% | 10.6% |
| Percent Waste lowmapq | 5.2% | 4.0% | 3.4% |
| Percent Waste Mitochondrial | 0.3% | 0.3% | 0.7% |
| Percent Waste No Barcode | 1.7% | 1.7% | 5.7% |
| Percent Waste Non Cell Barcode | 46.6% | 66.9% | 54.0% |
| Percent Waste Overall Nondup | 55.1% | 73.6% | 66.0% |
| Percent Waste Total | 68.7% | 81.3% | 76.6% |
| Percent Waste Unmapped | 0.7% | 0.4% | 1.6% |
| Median Fragments Per Cell | 10,746 | 7,700 | 13,310 |
| Median per Cell Unique Fragments at 30000 RRPC | 4,135.0 | 2,578.2 | 4,064.0 |
| Median per Cell Unique Fragments at 50000 RRPC | 6,501.4 | 4,095.3 | 6,437.5 |
| Num_Fragments | 6.13E+08 | 6.26E+08 | 2.73E+08 |
| r1_q30 Bases Percent | 94.4% | 94.3% | 86.6% |
| r2_q30 Bases Percent | 94.0% | 94.0% | 85.2% |
| si_q30 Bases Percent | 92.4% | 92.5% | 76.4% |
| Total Usable Fragments | 1.92E+08 | 1.17E+08 | 6.40E+07 |
| TSS Enrichment Score | 6.360 | 4.651 | 8.864 |

### REFERENCES

The following references are incorporated herein by reference in their entireties.
1 Basil, M. C. et al. The Cellular and Physiological Basis for Lung Repair and Regeneration: Past, Present, and Future. Cell Stem Cell 26, 482-502, doi:10.1016/j.stem.2020.03.009 PMID—32243808 (2020).
2 Barkauskas, C. E. et al. Type 2 alveolar cells are stem cells in adult lung. J Clin Invest 123, 3025-3036, doi:10.1172/jci68782 PMID-23921127 (2013).
3 Zacharias, W. J. et al. Regeneration of the lung alveolus by an evolutionarily conserved epithelial progenitor. Nature 555, 251-255, doi:10.1038/nature25786 PMID-29489752 (2018).
4 Toth, A., Zhao, B. & Zacharias, W. J. Lung Stem Cells in Development, Health and Disease. 122-133, doi:10.1183/2312508×.10009420 (2021).
5 Grant, R. A. et al. Circuits between infected macrophages and T cells in SARS-CoV-2 pneumonia. Nature, doi:10.1038/s41586-020-03148-w (2021).
6 Bharat, A. et al. Lung transplantation for patients with severe COVID-19. Sci Transl Med 12, doi:10.1126/scitranslmed.abe4282 (2020).
7 Barkauskas, C. E. et al. Lung organoids: current uses and future promise. 144, doi:10.1242/dev.140103 PMID-28292845 (2017).
8 Hawkins, F. et al. Prospective isolation of NKX2-1-expressing human lung progenitors derived from pluripotent stem cells. Journal of Clinical Investigation, doi:10.1172/JC189950 PMID-28463226 (2017).
9 Jacob, A. et al. Differentiation of Human Pluripotent Stem Cells into Functional Lung Alveolar Epithelial Cells. Cell Stem Cell 21, 472-488.e410, doi:10.1016/j.stem.2017.08.014 PMID-28965766 (2017).
10 Strikoudis, A. et al. Modeling of Fibrotic Lung Disease Using 3D Organoids Derived from Human Pluripotent Stem Cells. Cell Reports 27, 3709-3723 e3705, doi:10.1016/j.celrep.2019.05.077 PMID-31216486 (2019).
11 Kobayashi, Y. et al. Persistence of a regeneration-associated, transitional alveolar epithelial cell state in pulmonary fibrosis. Nature Cell Biology 22, 934-946, doi:10.1038/s41556-020-0542-8 PMID-32661339 (2020).
12 Salahudeen, A. A. et al. Progenitor identification and SARS-CoV-2 infection in human distal lung organoids. Nature 588, 670-675, doi:10.1038/s41586-020-3014-1 PMID-33238290 (2020).
13 Abo, K. M. et al. Human iPSC-derived alveolar and airway epithelial cells can be cultured at air-liquid interface and express SARS-CoV-2 host factors. Biorxiv, 2020.2006.2003.132639, doi:10.1101/2020.06.03.132639 PMID-32577635 (2020).
14 Barkauskas, C. E. et al. Type 2 alveolar cells are stem cells in adult lung. J Clin Invest 123, 3025-3036, doi:10.1172/JC168782 (2013).
15 Zepp, J. A. et al. Distinct Mesenchymal Lineages and Niches Promote Epithelial Self-Renewal and Myofibrogenesis in the Lung. Cell 170, 1134-1148 e1110, doi:10.1016/j.cell.2017.07.034 PMID-28886382 (2017).
16 Frank, D. B. et al. Emergence of a Wave of Wnt Signaling that Regulates Lung Alveologenesis by Controlling Epithelial Self-Renewal and Differentiation. Cell Reports 17, 2312-2325, doi:10.1016/j.celrep.2016.11.001 PMID-27880906 (2016).
17 Travaglini, K. J. et al. A molecular cell atlas of the human lung from single-cell RNA sequencing. Nature, 1-7, doi:10.1038/s41586-020-2922-4 PMID-33208946 (2020).
18 Nabhan, A. N., Brownfield, D. G., Harbury, P. B., Krasnow, M. A. & Desai, T. J. Single-cell Wnt signaling niches maintain stemness of alveolar type 2 cells. Science 359, 1118-1123, doi:10.1126/science.aam6603 PMID-29420258 (2018).
19 Riemondy, K. A. et al. Single cell RNA sequencing identifies TGFβ as a key regenerative cue following LPS-induced lung injury. Jci Insight 4, e123637, doi:10.1172/jci.insight.123637 PMID-30913038 (2019).
20 Choi, J. et al. Inflammatory Signals Induce AT2 Cell-Derived Damage-Associated Transient Progenitors that Mediate Alveolar Regeneration. Cell Stem Cell 27, 366-382.e367, doi:10.1016/j.stem.2020.06.020 PMID-32750316 (2020).
21 Strunz, M. et al. Alveolar regeneration through a Krt8⁺ transitional stem cell state that persists in human lung fibrosis. Nature Communications 11, 3559, doi:10.1038/s41467-020-17358-3 PMID-32678092 (2020).
22 Gibbs, C. S. et al. High-performance single-cell gene regulatory network inference at scale: the Inferelator 3.0. Bioinformatics 38, 2519-2528, doi:10.1093/bioinformatics/btac117 PMID-35188184 (2022).
23 Miraldi, E. R. et al. Leveraging chromatin accessibility for transcriptional regulatory network inference in T Helper 17 Cells. Genome Res 29, 449-463, doi:10.1101/gr.238253.118 PMID-30696696 (2019).
24 Little, D. R. et al. Differential chromatin binding of the lung lineage transcription factor NKX2-1 resolves opposing murine alveolar cell fates in vivo. Nature Communications 12, 2509, doi:10.1038/s41467-021-22817-6 PMID-33947861 (2021).
25 Ostrin, E. J. et al. β-Catenin maintains lung epithelial progenitors after lung specification. Development (Cambridge, England) 145, doi:10.1242/dev.160788 PMID-29440304 (2018).
26 Kusakabe, T. et al. Thyroid-specific enhancer-binding protein/NKX2.1 is required for the maintenance of ordered architecture and function of the differentiated thyroid. Mol Endocrinol 20, 1796-1809, doi:10.1210/me.2005-0327 (2006).
27 Dekkers, J. F. et al. High-resolution 3D imaging of fixed and cleared organoids. Nature Protocols 14, 1756-1771, doi:10.1038/s41596-019-0160-8 PMID-31053799 (2019).
28 Jain, R. et al. Plasticity of Hopx(+) type I alveolar cells to regenerate type II cells in the lung. Nat Commun 6, 6727, doi:10.1038/ncomms7727 (2015).
29 Hao, Y. et al. Integrated analysis of multimodal single-cell data. Cell 184, 3573-3587.e3529, doi:10.1016/j.cell.2021.04.048 (2021).
30 Sun, X. et al. A census of the lung: CellCards from LungMAP. Dev Cell 57, 112-145.e112, doi:https://doi.org/10.1016/j.devcel.2021.11.007 (2022).
31 Guo, M. et al. Single cell RNA analysis identifies cellular heterogeneity and adaptive responses of the lung at birth. Nature Communications 10, 37, doi:10.1038/s41467-018-07770-1 PMID-30604742 (2019).
32 Green, J., Endale, M., Auer, H. & Perl, A. K. Diversity of Interstitial Lung Fibroblasts Is Regulated by Platelet-Derived Growth Factor Receptor alpha Kinase Activity. Am J Respir Cell Mol Biol 54, 532-545, doi:10.1165/rcmb.2015-0095oc PMID-26414960 (2016).
33 Hamilton, T. G., Klinghoffer, R. A., Corrin, P. D. & Soriano, P. Evolutionary divergence of platelet-derived growth factor alpha receptor signaling mechanisms. Mol Cell Biol 23, 4013-4025, doi:10.1128/mcb.23.11.4013-4025.2003 (2003).
34 Jin, S. et al. Inference and analysis of cell-cell communication using CellChat. Nature Communications 12, 1088, doi:10.1038/s41467-021-21246-9 PMID-33597522 (2021).
35 Sucre, J. M. S. et al. Hyperoxia Injury in the Developing Lung is Mediated by Mesenchymal Expression of Wnt5A. Am J Resp Crit Care 0, 1249-1262, doi:10.1164/rccm.201908-1513oc PMID-32023086 (2019).
36 Negretti, N. M. et al. A single-cell atlas of mouse lung development. Development 148, doi:10.1242/dev.199512 PMID-34927678 (2021).
37 Paris, A. J. et al. STAT3-BDNF-TrkB signalling promotes alveolar epithelial regeneration after lung injury. Nature Cell Biology 22, 1197-1210, doi:10.1038/s41556-020-0569-x PMID-32989251 (2020).
38 Yuan, T., Volckaert, T., Chanda, D., Thannickal, V. J. & Langhe, S. P. D. Fgf10 Signaling in Lung Development, Homeostasis, Disease, and Repair After Injury. Frontiers Genetics 9, 418, doi:10.3389/fgene.2018.00418 PMID-30319693 (2018).
39 Lee, J. H. et al. Anatomically and Functionally Distinct Lung Mesenchymal Populations Marked by Lgr5 and Lgr6. Cell 170, 1149-1163 e1112, doi:10.1016/j.cell.2017.07.028 PMID-28886383 (2017).
40 Ruppert, C., Henneke, I., Korfei, M., Seeger, W. & Gunther, A. Role of HGF in the healthy and injured lung. European Respiratory Journal 46, PA3831, doi:10.1183/13993003.congress-2015.PA3831 (2015).
41 Street, K. et al. Slingshot: cell lineage and pseudotime inference for single-cell transcriptomics. Bmc Genomics 19, 477, doi:10.1186/s12864-018-4772-0. (2018).
42 Bergen, V., Lange, M., Peidli, S., Wolf, F. A. & Theis, F. J. Generalizing RNA velocity to transient cell states through dynamical modeling. Nature Biotechnology 38, 1408-1414, doi:10.1038/s41587-020-0591-3 PMID-32747759 (2020).
43 Gorin, G., Fang, M., Chari, T. & Pachter, L. RNA velocity unraveled. Biorxiv, 2022.2002.2012.480214, doi:10.1101/2022.02.12.480214 (2022).
44 Granja, J. M. et al. ArchR is a scalable software package for integrative single-cell chromatin accessibility analysis. Nature Genetics 53, 403-411, doi:10.1038/s41588-021-00790-6 (2021).
45 He, B. & Tan, K. Understanding transcriptional regulatory networks using computational models. Curr Opin Genet Dev 37, 101-108, doi:10.1016/j.gde.2016.02.002 (2016).
46 Duren, Z., Chen, X., Jiang, R., Wang, Y. & Wong, W. H. Modeling gene regulation from paired expression and chromatin accessibility data. Proc National Acad Sci 114, E4914-E4923, doi:10.1073/pnas.1704553114 PMID-28576882 (2017).
47 Cardenas-Diaz, F. L. et al. Temporal and spatial staging of lung alveolar regeneration is determinedby the grainyhead transcription factor<em>Tfcp211</em>. Biorxiv, 2022.2008.2023.504977, doi:10.1101/2022.08.23.504977 (2022).
48 Chung, M. I., Bujnis, M., Barkauskas, C. E., Kobayashi, Y. & Hogan, B. L. M. Niche-mediated BMP/SMAD signaling regulates lung alveolar stem cell proliferation and differentiation. Development 145, dev163014, doi:10.1242/dev.163014 PMID-29752282 (2018).
49 Serra, M. et al. Pluripotent stem cell differentiation reveals distinct developmental pathways regulating lung-versus thyroid-lineage specification. Development 144, 3879-3893, doi:10.1242/dev.150193 PMID-28947536 (2017).
50 Gaskill, C. F. et al. Disruption of lineage specification in adult pulmonary mesenchymal progenitor cells promotes microvascular dysfunction. Journal of Clinical Investigation, doi:10.1172/JCI88629 PMID-28463231 (2017).
51 Flodby, P. et al. Cell-specific expression of aquaporin-5 (Aqp5) in alveolar epithelium is directed by GATA6/Spl via histone acetylation. Sci Rep-uk 7, 3473, doi:10.1038/s41598-017-03152-7 PMID-28615712 (2017).
52 Chung, C. et al. Hippo-Foxa2 signaling pathway plays a role in peripheral lung maturation and surfactant homeostasis. Proc National Acad Sci 110, 7732-7737, doi:10.1073/pnas.1220603110 PMID-23620511 (2013).
53 Herriges, M. J. et al. Long noncoding RNAs are spatially correlated with transcription factors and regulate lung development. 28, doi:10.1101/gad.238782.114 PMID 24939938.
54 LaCanna, R. et al. Yap/Taz regulate alveolar regeneration and resolution of lung inflammation. J Clin Invest 130, 2107-2122, doi:10.1172/jci125014 PMID-30985294 (2019).
55 Gokey, J. J. et al. Active epithelial Hippo signaling in idiopathic pulmonary fibrosis. Jci Insight 3, doi:10.1172/jci.insight.98738 PMID-29563341 (2018).
56 Finn, J. et al. Dlkl-Mediated Temporal Regulation of Notch Signaling Is Required for Differentiation of Alveolar Type II to Type I Cells during Repair. Cell Reports 26, 2942-2954.e2945, doi:10.1016/j.celrep.2019.02.046 PMID-30865885 (2019).
57 Carvalho, A. d. et al. Glycogen synthase kinase 3 induces multilineage maturation of human pluripotent stem cell-derived lung progenitors in 3D culture. Development 146, dev171652, doi:10.1242/dev.171652 PMID-30578291 (2019).
58 Jain, R. et al. Plasticity of Hopx(+) type I alveolar cells to regenerate type II cells in the lung. Nat Commun 6, 6727, doi:10.1038/ncomms7727 PMID-25865356 (2015).
59 Yang, Y. et al. Transcription Factor C/EBP Homologous Protein in Health and Diseases. Front Immunol 8, 1612, doi:10.3389/fimmu.2017.01612 PMID-29230213 (2017).
60 Beers, M. F. et al. Alveolar Type 2 Epithelial Cell Quality Control Responses to Pulmonary Fibrosis Related SFTPC Mutations Are Dysfunctional And Substrate Specific. Faseb J 34, 1-1, doi:10.1096/fasebj.2020.34.sl.05725 (2020).
61 Weng, A. et al. Lung Injury Induces Alveolar Type 2 Cell Hypertrophy and Polyploidy with Implications for Repair and Regeneration. Am J Resp Cell Mol 66, 564-576, doi:10.1165/rcmb.2021-0356oc PMID-35202558 (2022).
62 Wiel, C. et al. BACH1 Stabilization by Antioxidants Stimulates Lung Cancer Metastasis. Cell 178, 330-345.e322, doi:10.1016/j.cell.2019.06.005 PMID-31257027 (2019).
63 Lignitto, L. et al. Nrf2 Activation Promotes Lung Cancer Metastasis by Inhibiting the Degradation of Bachl. Cell 178, 316-329.e318, doi:10.1016/j.cell.2019.06.003 PMID-31257023 (2019).
64 Liang, W., Guo, L., Liu, T. & Qin, S. MEF2C alleviates acute lung injury in cecal ligation and puncture (CLP)-induced sepsis rats by up-regulating AQP1. Allergol Immunopath 49, 117-124, doi:10.15586/aei.v49i5.477 PMID-34476932 (2021).
65 Zhou, B. et al. Comprehensive epigenomic profiling of human alveolar epithelial differentiation identifies key epigenetic states and transcription factor co-regulatory networks for maintenance of distal lung identity. Bmc Genomics 22, 906, doi:10.1186/s12864-021-08152-6 PMID-34922464 (2021).
66 Lieshout, L. P. v. et al. A Novel Triple-Mutant AAV6 Capsid Induces Rapid and Potent Transgene Expression in the Muscle and Respiratory Tract of Mice. Mol Ther—Methods Clin Dev 9, 323-329, doi:10.1016/j.omtm.2018.04.005 PMID-30038936 (2018).
67 Rindler, T. N. et al. Efficient Transduction of Alveolar Type 2 Cells with Adeno-associated Virus for the Study of Lung Regeneration. Am J Resp Cell Mol 65, 118-121, doi:10.1165/rcmb.2021-00491e PMID-34241584 (2021).
68 Srinivas, S. et al. Cre reporter strains produced by targeted insertion of EYFP and ECFP into the ROSA26 locus. BMC Dev Biol 1, 4, doi:10.1186/1471-213x-1-4 (2001).
69 Maeda, Y. et al. Kras(G12D) and Nkx2-1 haploinsufficiency induce mucinous adenocarcinoma of the lung. The Journal of clinical investigation 122, 4388-4400, doi:10.1172/JC164048 PMID-23143308 (2012).
70 Mollaoglu, G. et al. The Lineage-Defining Transcription Factors SOX2 and NKX2-1 Determine Lung Cancer Cell Fate and Shape the Tumor Immune Microenvironment. Immunity 49, 764-779.e769, doi:10.1016/j.immuni.2018.09.020 PMID-30332632 (2018).
71 Tata, P. R. et al. Developmental History Provides a Roadmap for the Emergence of Tumor Plasticity. Dev Cell 44, 679-693.e675, doi:10.1016/j.devcel.2018.02.024 PMID-29587142 (2018).
72 Laughney, A. M. et al. Regenerative lineages and immune-mediated pruning in lung cancer metastasis. Nature Medicine 26, 259-269, doi:10.1038/s41591-019-0750-6 (2020).
73 Han, L. et al. Single cell transcriptomics identifies a signaling network coordinating endoderm and mesoderm diversification during foregut organogenesis. Nature Communications 11, 4158, doi:10.1038/s41467-020-17968-x (2020).
74 Maruyama, E. O. et al. Cell-Specific Cre Strains For Genetic Manipulation in Salivary Glands. Plos One 11, e0146711, doi:10.1371/journal.pone.0146711 (2016).
75 Rodriguez-Castillo, J. A. et al. Understanding alveolarization to induce lung regeneration. Respiratory Research 19, 148, doi:10.1186/s12931-018-0837-5 (2018).
76 Barnes, P. J., Anderson, G. P., Fageras, M. & Belvisi, M. G. Chronic lung diseases: prospects for regeneration and repair. European Respiratory Review 30, 200213, doi:10.1183/16000617.0213-2020 (2021).
77 Kathiriya, J. J. et al. Human alveolar type 2 epithelium transdifferentiates into metaplastic KRT5⁺ basal cells. Nature Cell Biology 24, 10-23, doi:10.1038/s41556-021-00809-4 PMID-34969962 (2022).
78 Gorin, G., Svensson, V. & Pachter, L. Protein velocity and acceleration from single-cell multiomics experiments. Genome Biology 21, 39, doi:10.1186/s13059-020-1945-3 PMID-32070398 (2020).
79 Yamaguchi, T., Hosono, Y., Yanagisawa, K. & Takahashi, T. NKX2-1/TTF-1: an enigmatic oncogene that functions as a double-edged sword for cancer cell survival and progression. Cancer Cell 23, 718-723, doi:10.1016/j.ccr.2013.04.002 (2013).
80 Verheyden, J. M. & Sun, X. A transitional stem cell state in the lung. Nature Cell Biology 22, 1025-1026, doi:10.1038/s41556-020-0561-5 (2020).
81 Ikegami, M., Na, C. L., Korfhagen, T. R. & Whitsett, J. A. Surfactant protein D influences surfactant ultrastructure and uptake by alveolar type II cells. Am J Physiol Lung Cell Mol Physiol 288, L552-561, doi:10.1152/ajplung.00142.2004 (2005).
82 Manno, L. G. et al. RNA velocity of single cells. Nature 560, 494-498, doi:10.1038/s41586-018-0414-6 PMID-30089906 (2018).
83 McGinnis, C. S., Murrow, L. M. & Gartner, Z. J. DoubletFinder: Doublet Detection in Single-Cell RNA Sequencing Data Using Artificial Nearest Neighbors. Cell Systems 8, 329-337.e324, doi:10.1016/j.cels.2019.03.003 PMID-30954475 (2019).
84 Lange, M. et al. CellRank for directed single-cell fate mapping. Nature Methods 19, 159-170, doi:10.1038/s41592-021-01346-6 (2022).
85 Katsura, H. et al. Human lung stem cell-based alveolospheres provide insights into SARS-CoV-2 mediated interferon responses and pneumocyte dysfunction. Cell Stem Cell, doi:10.1016/j.stem.2020.10.005 PMID-33128895 (2020).
86 Venema, W. T. C. U. et al. Gut mucosa dissociation protocols influence cell type proportions and single-cell gene expression levels. Sci Rep-uk 12, 9897, doi:10.1038/s41598-022-13812-y PMID-35701452 (2022).
87 Llufrio, E. M., Wang, L., Naser, F. J. & Patti, G. J. Sorting cells alters their redox state and cellular metabolome. Redox Biol 16, 381-387, doi:10.1016/j.redox.2018.03.004 PMID-29627745 (2018).
88 Box, A. et al. Evaluating the Effects of Cell Sorting on Gene Expression. J Biomol Techniques Jbt 31, 100-111, doi:10.7171/jbt.2020-3103-004 PMID-32831654 (2020).
89 Binek, A. et al. Flow Cytometry Has a Significant Impact on the Cellular Metabolome. J Proteome Res 18, 169-181, doi:10.1021/acs.jproteome.8b00472 PMID-30362351 (2019).
90 Pfister, G., Toor, S. M., Nair, V. S. & Elkord, E. An evaluation of sorter induced cell stress (SICS) on peripheral blood mononuclear cells (PBMCs) after different sort conditions—Are your sorted cells getting SICS?J Immunol Methods 487, 112902, doi:10.1016/j.jim.2020.112902 PMID-33069766 (2020).
91 Neuschulz, A. et al. A single-cell RNA labeling strategy for measuring stress response upon tissue dissociation. Mol Syst Biol 19, e11147, doi:10.15252/msb.202211147 PMID-36573354 (2023).
92 Dolinay, T., Himes, B. E., Shumyatcher, M., Lawrence, G. G. & Margulies, S. S. Integrated Stress Response Mediates Epithelial Injury in Mechanical Ventilation. American Journal of Respiratory Cell and Molecular Biology 57, 193-203, doi:10.1165/rcmb.2016-0404oc PMID-28363030 (2017).
93 Wu, H. et al. Progressive Pulmonary Fibrosis Is Caused by Elevated Mechanical Tension on Alveolar Stem Cells. Cell 180, 107-121.e117, doi:10.1016/j.cell.2019.11.027 PMID-31866069 (2020).
94 Du, Y., Guo, M., Whitsett, J. A. & Xu, Y. ‘LungGENS’: a web-based tool for mapping single-cell gene expression in the developing lung. Thorax 70, 1092-1094, doi:10.1136/thoraxjnl-2015-207035 (2015).
95 Du, Y. et al. Lung Gene Expression Analysis (LGEA): an integrative web portal for comprehensive gene expression data analysis in lung development. Thorax 72, 481-484, doi:10.1136/thoraxjnl-2016-209598 (2017).
96 Iwafuchi-Doi, M. & Zaret, K. S. Pioneer transcription factors in cell reprogramming. Genes Dev 28, 2679-2692, doi:10.1101/gad.253443.114 (2014).
97 Mayran, A. & Drouin, J. Pioneer transcription factors shape the epigenetic landscape. Journal of Biological Chemistry 293, 13795-13804, doi:https://doi.org/10.1074/jbc.R117.001232 (2018).
98 Kligerman, S. J., Franks, T. J. & Galvin, J. R. From the radiologic pathology archives: organization and fibrosis as a response to lung injury in diffuse alveolar damage, organizing pneumonia, and acute fibrinous and organizing pneumonia. 33, doi:10.1148/rg.337130057 PMID-24224590.
99 Li, R., Li, X., Hagood, J., Zhu, M. S. & Sun, X. Myofibroblast contraction is essential for generating and regenerating the gas-exchange surface. J Clin Invest 130, 2859-2871, doi:10.1172/jci132189 (2020).
100 Zepp, J. A. et al. Genomic, epigenomic, and biophysical cues controlling the emergence of the lung alveolus. Science 371, doi:10.1126/science.abc3172 (2021).

What is claimed is:

1. A method of making an alveolar epithelial progenitor cell (AEP)-derived organoid (AEP-O), the method comprising coculturing Wnt-responsive alveolar type 2 cells (AEP cells) and mesenchyme cells.

2. The method of claim 1, wherein the mesenchyme cells are fibroblast cells.

3. The method of any one of the preceding claims, wherein the mesenchyme cells are alveolar fibroblasts.

4. The method of any one of the preceding claims, wherein the mesenchyme cells are from P28 wild type C57BL/6 mice, optionally at passage 3-4.

5. The method of any one of the preceding claims, wherein the AEP cells are FACS sorted to select CD31⁻/CD45⁻/CD326⁺ (EpCAM⁺) cells, wherein optionally the AEP cells are TdTomato⁺.

6. The method of any one of the preceding claims, wherein the AEP cells are from Axin2^{creERT2-tDT} mice.

7. The method of any one of the preceding claims, wherein the AEP and mesenchyme cells are cocultured in a ratio of AEP to mesenchyme that is, or is about, 2:1, 1:1, 1:2, 1:5, 1:10, 1:15, 1:20, 1:50, or a range defined by any two of the preceding values, optionally 2:1-1:50, 1:2-1:20, 1:5-1:15, or 1:10.

8. The method of any one of the preceding claims, wherein about 1,000, 2,000, 3,000, 4,000, 5,000, 6,000, 7,000, 8,000, 9,000, or 10,000, or a range defined by any two of the preceding values, of sorted AEPs are cocultured with about 10K, 20K, 30K, 40K, 50K, 60, 70K, 80K, 90K, or 100K, or a range defined by any two of the preceding values, mesenchymal cells.

9. The method of any one of the preceding claims, wherein the coculturing is in a small airway epithelial cell growth basal medium (Lonza, CC-3119) or equivalent media.

10. The method of any one of the preceding claims, wherein the coculturing is in a media supplemented with BPE, Insulin, Retinoic Acid, Transferrin, and hEGF.

11. The method of any one of the preceding claims, wherein the coculturing is in a media supplemented with heat inactivated fetal bovine serum, optionally at a final concentration of about 1-10%, 2-8%, 3-7%, or 5%.

12. The method of any one of the preceding claims, wherein the coculturing is in a media comprising an extracellular membrane matrix.

13. The method of any one of the preceding claims, wherein the coculturing is in a media comprising Matrigel.

14. The method of any one of the preceding claims, wherein the coculturing is in a media comprising an extracellular membrane matrix, wherein the media and the extracellular membrane matrix are combined in a ratio of 10:1, 5:1, 3:1, 2:1, 1:1, 1:2, 1:3, 1:5, or 1:10, or a range defined by any two of the preceding values.

15. The method of any one of the preceding claims, wherein the coculturing is in a transwell.

16. The method of any one of the preceding claims, wherein the coculturing is in a transwell, wherein media supplemented with a ROCK inhibitor is added beneath the transwell.

17. The method of any one of the preceding claims, wherein the coculturing is in a transwell, wherein media supplemented with ROCK Inhibitor Y-27632 dihydrochloride is added beneath the transwell, optionally at a final concentration of about 0.001-0.1, 0.005-0.05, or 0.01 mM.

18. The method of any one of the preceding claims, wherein the coculturing is in a transwell, wherein media supplemented with a ROCK inhibitor is added beneath the transwell, and wherein the coculture is incubated in the presence of the ROCK inhibitor for about 36-60, or 48 hours.

19. The method of any one of the preceding claims, wherein the coculturing is in a transwell, wherein media supplemented with a ROCK inhibitor is added beneath the transwell, and wherein the coculture is incubated in the presence of the ROCK inhibitor for about 36-60, or 48 hours, and wherein thereafter the media does not contain a ROCK inhibitor.

20. The method of any one of the preceding claims, wherein the coculturing is for a period of, or of at least, about 1, 2, 3, 4, 5, 6, 7, or 8 weeks, or a range defined by any two of the preceding values, optionally 1-8, 2-8, 3-8, 2-6, 2-5, or 3-5 weeks.

21. The method of any one of the preceding claims, wherein the AEP-O comprises AEP cells, AT1 cells, and AT2 cells, optionally wherein the AEP, AT1 and AT2 cells express AEP, AT1, and AT2 cell markers, respectively.

22. The method of any one of the preceding claims, wherein the AEP-O comprises RAGE⁺ AT1 cells.

23. The method of any one of the preceding claims, wherein the AEP-O comprises cavities.

24. The method of any one of the preceding claims, wherein the AEP-O comprises cavities forming alveolar-like structures.

25. The method of any one of the preceding claims, wherein the AEP-O comprises mature and/or polarized AT1 cells within the central portion of the organoid, optionally wherein AT2 cells are intermixed with the mature and/or polarized AT1 cells.

26. The method of any one of the preceding claims, wherein the AEP-O comprises cavities with epithelial lining that comprises AT2 cells.

27. The method of any one of the preceding claims, wherein the AEP-O comprises cavities with epithelial lining that comprises AT2 cells containing lamellar bodies with the apical surface directed towards the internal lumen.

28. The method of any one of the preceding claims, wherein the AEP-O comprises active surfactant secretion.

29. The method of any one of the preceding claims, wherein the AEP-O comprises mesenchymal cells, optionally fibroblasts.

30. The method of any one of the preceding claims, wherein the AEP-O does not comprise mesenchymal cells, optionally fibroblasts.

31. The method of any one of the preceding claims, wherein the AEP-O does not comprise fibillar collagen type I and/or type II.

32. The method of any one of the preceding claims, wherein mesenchymal cells, optionally fibroblasts, are near and/or adjacent to the AEP-O.

33. The method of any one of the preceding claims, wherein the AEP-O is cultured in a container comprising a monolayer of mesenchymal cells, optionally fibroblasts.

34. The method of any one of the preceding claims, wherein the AEP-O comprises immune cells.

35. The method of any one of the preceding claims, wherein the AEP-O does not comprise immune cells.

36. The method of any one of the preceding claims, wherein immune cells are near and/or adjacent to the AEP-O.

37. The method of any one of the preceding claims, wherein the AEP-O are cultured in a container comprising a monolayer of fibroblasts comprising immune cells.

38. The method of any one of the preceding claims, wherein the AEP-O comprises AT1 cells expressing WNT ligands and/or PDGF ligands.

39. The method of any one of the preceding claims, wherein the AEP-O comprises AT2 cells that are WNT-responsive.

40. The method of any one of the preceding claims, wherein the mesenchymal cells are WNT-responsive, PDGFRα⁺, express HGF, express non-canonical WNT, and/or express FGF ligands.

41. The method of any one of the preceding claims, wherein the AEP-O comprises AEP cells expressing one or more of the AEP-enriched markers Id2, Ctnnb1, Lrp5, Lrp2, Napsa, Bex2, Hdc, and Fgfr2.

42. The method of any one of the preceding claims, wherein the AEP-O comprises AEP cells expressing high levels of cycle genes (pAEPs).

43. The method of any one of the preceding claims, wherein the AEP-O comprises AT2tr cells, optionally comprising high level expression of glutathione pathway genes and a shift towards lipid metabolism.

44. The method of any one of the preceding claims, wherein the AEP-O comprises mature AT2 cells expressing one or more markers selected from Sftpa1, Lys2, Sftpc and Sftpb.

45. The method of any one of the preceding claims, wherein the AEP-O comprises Krt8⁺ transition cells (Krt8⁺) expressing one or more markers selected from Krt8, Lgals3, Tp53, Nupr1, Ddit3, and Cldn4, or optionally expressing one or more markers selected from Krt8, Lgals3, Tp53, and Cldn4.

46. The method of any one of the preceding claims, wherein the AEP-O comprises AT1 transition (AT1tr) cells expressing one or more markers selected from Hes1 and Igfbp7.

47. The method of any one of the preceding claims, wherein the AEP-O comprises a modification reducing or eliminating expression of Nkx2-1.

48. The method of any one of the preceding claims, wherein the AEP-O comprises AEPs harboring a R26R-lox-stop-lox-EYFP allele.

49. The method of any one of the preceding claims, wherein the method comprises infecting AEPs with AAV6.2FF-Cre, optionally after FACS sorting and prior to coculturing with mesenchyme cells.

50. The method of any one of the preceding claims, wherein the AEPs are from Axin2^{creERT2-Tdt}×Rosa-EYFP× Nkx2-1^{flox/flox} animals.

51. The method of any one of the preceding claims, wherein the AEPs are from Tfcp211^{CreERT2}×R26R^{EYFP}×Nkx2-1^{flox/flox}×animals.

52. The method of any one of the preceding claims, wherein the AEPs are from Nkx2-1 knockout animals.

53. The method of any one of the preceding claims, wherein the AEP-O comprises at least one alveolar-like cavity filled with debris, a pseudostratrified epithelial lining, and/or a glandular-like appearance.

54. The method of any one of the preceding claims, wherein the AEP-O does not comprise substantial expression of one or more foregut endoderm markers selected from Sox2, Sox9, Cdx2, Gata4, and Pdx1.

55. An AEP-O made by the method of any one of the preceding claims.

56. An AEP-O comprising AEP cells, AT1 cells, and AT2 cells, optionally wherein the AEP, AT1 and AT2 cells express AEP, AT1, and AT2 cell markers, respectively.

57. The AEP-O of any one of the preceding claims, wherein the AEP-O comprises RAGE⁺ AT1 cells.

58. The AEP-O of any one of the preceding claims, wherein the AEP-O comprises cavities.

59. The AEP-O of any one of the preceding claims, wherein the AEP-O comprises cavities forming alveolar-like structures.

60. The AEP-O of any one of the preceding claims, wherein the AEP-O comprises mature and/or polarized AT1 cells within the central portion of the organoid, optionally wherein AT2 cells are intermixed with the mature and/or polarized AT1 cells.

61. The AEP-O of any one of the preceding claims, wherein the AEP-O comprises cavities with epithelial lining that comprises AT2 cells.

62. The AEP-O of any one of the preceding claims, wherein the AEP-O comprises cavities with epithelial lining that comprises AT2 cells containing lamellar bodies with the apical surface directed towards the internal lumen.

63. The AEP-O of any one of the preceding claims, wherein the AEP-O comprises active surfactant secretion.

64. The AEP-O of any one of the preceding claims, wherein the AEP-O comprises mesenchymal cells, optionally fibroblasts.

65. The AEP-O of any one of the preceding claims, wherein the AEP-O does not comprise mesenchymal cells, optionally fibroblasts.

66. The AEP-O of any one of the preceding claims, wherein the AEP-O does not comprise fibillar collagen type I and/or type II.

67. The AEP-O of any one of the preceding claims, wherein mesenchymal cells, optionally fibroblasts, are near and/or adjacent to the AEP-O.

68. The AEP-O of any one of the preceding claims, wherein the AEP-O is cultured in a container comprising a monolayer of mesenchymal cells, optionally fibroblasts.

69. The AEP-O of any one of the preceding claims, wherein the AEP-O comprises immune cells.

70. The AEP-O of any one of the preceding claims, wherein the AEP-O does not comprise immune cells.

71. The AEP-O of any one of the preceding claims, wherein immune cells are near and/or adjacent to the AEP-O.

72. The AEP-O of any one of the preceding claims, wherein the AEP-O are cultured in a container comprising a monolayer of fibroblasts comprising immune cells.

73. The AEP-O of any one of the preceding claims, wherein the AEP-O comprises AT1 cells expressing WNT ligands and/or PDGF ligands.

74. The AEP-O of any one of the preceding claims, wherein the AEP-O comprises AT2 cells that are WNT-responsive.

75. The AEP-O of any one of the preceding claims, wherein the mesenchymal cells are WNT-responsive, PDGFRα⁺, express HGF, express non-canonical WNT, and/or express FGF ligands.

76. The AEP-O of any one of the preceding claims, wherein the AEP-O comprises AEP cells expressing one or more of the AEP-enriched markers Id2, Ctnnb1, Lrp5, Lrp2, Napsa, Bex2, Hdc, and Fgfr2.

77. The AEP-O of any one of the preceding claims, wherein the AEP-O comprises AEP cells expressing high levels of cycle genes (pAEPs).

78. The AEP-O of any one of the preceding claims, wherein the AEP-O comprises AT2tr cells, optionally comprising high level expression of glutathione pathway genes and a shift towards lipid metabolism.

79. The AEP-O of any one of the preceding claims, wherein the AEP-O comprises mature AT2 cells expressing one or more markers selected from Sftpa1, Lys2, Sftpc and Sftpb.

80. The AEP-O of any one of the preceding claims, wherein the AEP-O comprises Krt8⁺ transition cells (Krt8⁺) expressing one or more markers selected from Krt8, Lgals3, Tp53, Nupr1, Ddit3, and Cldn4, or optionally expressing one or more markers selected from Krt8, Lgals3, Tp53, and Cldn4.

81. The AEP-O of any one of the preceding claims, wherein the AEP-O comprises AT1 transition (AT1tr) cells expressing one or more markers selected from Hes1 and Igfbp7.

82. The AEP-O of any one of the preceding claims, wherein the AEP-O comprises a modification reducing or eliminating expression of Nkx2-1.

83. The AEP-O of any one of the preceding claims, wherein the AEP-O comprises AEPs harboring a R26R-lox-stop-lox-EYFP allele.

84. The AEP-O of any one of the preceding claims, wherein the AEPs are infected with AAV6.2FF-Cre.

85. The AEP-O of any one of the preceding claims, wherein the AEPs are from Axin2^{CreERT2-Tdt}×Rosa-EYFP× Nkx2^{flox/flox} animals.

86. The AEP-O of any one of the preceding claims, wherein the AEPs are from Tfcp211^{CreERT2}×R26R^{EYFP}×Nkx2-1^{flox/flox} animals.

87. The AEP-O of any one of the preceding claims, wherein the AEPs are from Nkx2-1 knockout animals.

88. The AEP-O of any one of the preceding claims, wherein the AEP-O comprises at least one alveolar-like cavity filled with debris, a pseudostratrified epithelial lining, and/or a glandular-like appearance.

89. The AEP-O of any one of the preceding claims, wherein the AEP-O does not comprise substantial expression of one or more foregut endoderm markers selected from Sox2, Sox9, Cdx2, Gata4, and Pdx1.

90. The AEP-O of any one of the preceding claims, wherein the AEP cells are from Axin2^{creERT2-tDT} mice.

91. The AEP-O of any one of the preceding claims, wherein the AEP-O is embedded in an extracellular matrix.

92. The AEP-O of any one of the preceding claims, wherein the AEP-O is embedded in Matrigel.

93. The AEP-O of any one of the preceding claims, wherein the AEP-O is a model for a disease state.

94. The AEP-O of any one of the preceding claims, wherein the AEP-O has a genetic modification, optionally wherein the modification induces a model disease state.

95. A method comprising exposing an AEP-O of any one of the preceding claims to a compound.

96. The method of claim 95, wherein the compound is selected from a therapeutic compound, a candidate therapeutic compound, a toxin, mutagen, and/or a compound that induces a disease-like state in the AEP-O.

97. The method of claim 95 or 96, wherein the method comprises screening multiple compounds and/or multiple AEP-Os.

98. The method of any one of claims 95-97, wherein the method further comprises assessing a response of the AEP-O to exposure to compound.
